(19) United States
(12) Patent Application Publication
(10) Pub. No.: US 2024/0189424 A1
(43) Pub. Date: Jun. 13, 2024

(54) ENGINEERED NATURAL LIGAND-BASED CAR: DIRECTED EVOLUTION OF THE STRESS-RECEPTOR NKP30

(71) Applicant: THE TRUSTEES OF DARTMOUTH COLLEGE, Hanover, NH (US)

(72) Inventors: Savannah E. BUTLER, Hanover, NH (US); Rachel A. BROG, Hanover, NH (US); Cheryl H. CHANG, Hanover, NH (US); Charles L. SENTMAN, Grantham, NH (US); Yina H. HUANG, Hanover, NH (US); Margaret E. ACKERMAN, Hanover, NH (US)

(21) Appl. No.: 18/364,503

(22) Filed: Aug. 3, 2023

Related U.S. Application Data
(63) Continuation of application No. PCT/US2022/015214, filed on Feb. 4, 2022.
(60) Provisional application No. 63/145,554, filed on Feb. 4, 2021.

(51) Int. Cl. A61K 39/00; A61K 45/06; A61K 47/64; A61K 47/68; A61P 35/00; C07K 14/705; C07K 14/725; C07K 16/28

(52) U.S. Cl. CPC A61K 39/464411; A61K 39/4611; A61K 39/4631; A61K 39/4633; A61K 45/06; A61K 47/6425; A61K 47/6849; A61P 35/00; C07K 14/70503; C07K 14/7051; C07K 16/2809; C07K 16/2818; C07K 16/2827; C07K 2319/30

(57) ABSTRACT

Directed evolution using yeast display was employed to isolate novel NKp30 variants that bind to B7H6 with higher affinity compared to the native receptor but retain its fast association and dissociation profile. Two variants, CC3 and CC5, were expressed as soluble Fc-fusion proteins and CARs containing CD28 and CD3ζ intracellular domains. These Fc fusion protein forms of NKp30 and its variants were better able to bind tumor cells expressing low levels of B7H6 than TZ47, and exhibited improved in vitro tumor cell killing relative to NKp30. Also, CAR T cells expressing the engineered variants produced unique cytokine signatures in response to multiple tumor types expressing B7H6 compared to both NKp30 and TZ47. These findings suggest that natural CAR receptors can be fine-tuned to produce more desirable signaling outputs while maintaining evolutionary advantages in ligand recognition relative to scFvs.

The present application claims priority to Provisional Application No. 63/145,554 filed on Feb. 4, 2021 the contents of which are incorporated by reference in its entirety herein.

### FIELD OF INVENTION

The present disclosure relates to modified or variant NKp30 polypeptides, NKp30 variant comprising fusion polypeptides, optionally Fc fusion polypeptides, NKp30 variant comprising conjugates, NKp30 variant comprising bispecific T cell engagers or NKp30 variant comprising CARs, nucleic acids encoding any of the foregoing, ADCs comprising said modified or variant NKp30 polypeptides and a drug, bispecific T cell engagers and CARs which comprise said modified or variant NKp30 polypeptides and nucleic acids encoding, cells which express said modified NKp30 polypeptides and ADCs, bispecific T cell engagers and CARs comprising same, pharmaceutical and diagnostic compositions comprising any of the foregoing and methods of use of any of the foregoing in therapy and diagnosis, e.g., for the treatment of cancer and infectious conditions wherein the disease involves cells which express or overexpress one or more NKp30 ligands optionally B7H6 or BAT3.

### BACKGROUND OF THE INVENTION

The global burden of cancer doubled between 1975 and 2000, and cancer is expected to become the leading cause of death worldwide by 2010. According to the American Cancer Society, it is projected to double again by 2020 and to triple by 2030. Thus, there is a need for more effective therapies to treat various forms of cancer. Ideally, any cancer therapy should be effective (at killing cancerous cells), targeted (i.e. selective, to avoid killing healthy cells), permanent (to avoid relapse and metastasis), and affordable. Today's standards of care for most cancers fall short in some or all of these criteria.

T cells, especially cytotoxic T cells, play important roles in anti-tumor immunity (Rossing and Brenner (2004) Mol. Ther. 10:5-18). Adoptive transfer of tumor-specific T cells into patients provides a means to treat cancer (Sadelain, et al. (2003) Nat. Rev. Cancer 3:35-45). However, the traditional approaches for obtaining large numbers of tumor-specific T cells are time-consuming, laborious and sometimes difficult because the average frequency of antigen-specific T cells in periphery is extremely low (Rosenberg (2001) Nature 411:380-384; Ho, et al. (2003) Cancer Cell 3:431-437; Crowley, et al. (1990) Cancer Res. 50:492-498). In addition, isolation and expansion of T cells that retain their antigen specificity and function can also be a challenging task (Sadelain, et al. (2003) supra). Genetic modification of primary T cells with tumor-specific immunoreceptors, such as full-length T cell receptors or chimeric T cell receptor molecules can be used for redirecting T cells against tumor cells (Stevens, et al. (1995) J. Immunol. 154:762-771; Oelke, et al. (2003) Nat. Med. 9:619-624; Stancovski, et al. (1993) J. Immunol. 151:6577-6582; Clay, et al. (1999) J. Immunol. 163:507-153). This strategy avoids the limitation of low frequency of antigen-specific T cells, allowing for facilitated expansion of tumor-specific T cells to therapeutic doses.

Natural killer (NK) cells are innate effector cells serving as a first line of defense against certain viral infections and tumors (Biron, at al. (1999) Annu. Rev. Immunol. 17:189-220; Trinchieri (1989) Adv. Immunol. 47:187-376). They have also been implicated in the rejection of allogeneic bone marrow transplants (Lanier (1995) Curr. Opin. Immunol. 7:626-631; Yu, et al. (1992) Annu. Rev. Immunol. 10:189-214). Innate effector cells recognize and eliminate their targets with fast kinetics, without prior sensitization. Therefore, NK cells need to sense if cells are transformed, infected, or stressed to discriminate between abnormal and healthy tissues. According to the missing self phenomenon (Karre, et al. (1986) Nature (London) 319:675-678), NK cells accomplish this by looking for and eliminating cells with aberrant major histocompatibility complex (MHC) class I expression; a concept validated by showing that NK cells are responsible for the rejection of the MHC class I-deficient lymphoma cell line RMA-S, but not its parental MHC class I-positive line RMA.

Natural killer (NK) cells can also attack tumor and virally infected cells in the absence of MHC restriction, utilizing a combination of signals from activating and inhibitory receptors. One group of activating NK receptors are natural cytotoxicity receptors (NCRs), which include NKp46 (NCR1), NKp44 (NCR2) and NKp30 (also called natural cytotoxicity receptor 3 (NCR3) or CD337). These receptors are exclusively expressed on NK cells, which play important roles in NK-mediated tumor cell-killing.

NKp30 is an activating NK receptor that is involved in the NK-mediated killing of tumor cells. NKp30 recognizes ligands on tumor cells and dendritic cells. These ligands are highly expressed on a subset of tumor cells, but not most other normal cells. There is some evidence that some subsets of dendritic cells may express these ligands in vitro. In laboratory mice, NKp30 is a pseudogene. NKp30 has been further described in the literature including Brandt et al., J Exp Med. 2009 Jul. 6; 206(7):1495-503; Byrd et al., PLoS One. 2007 Dec. 19; 2(12):e1339; and Delahaye et al., Nat Med. 2011 June; 17(6):700-7, each of which is incorporated by reference herein in its entirety.

Different cellular NKp30 receptor ligands have been identified including e.g., BAT3 and B7-H6. BAT3 is a nuclear protein, which is involved in the interaction with P53 and induction of apoptosis after stress such as DNA damage. B7-H6 is a B7 family member. The structures of both BAT3 and B&H6 are known. Additionally, the structures of an NKp30 ligand binding site and an NKp30-B7-H6 complex have been reported in the literature (Li et al., J Exp Med. 2011 Apr. 11; 208(4):703-14; Joyce et al., Proc Natl Acad Sci USA. 2011 Apr. 12; 108(15):6223-8).

Unlike BAT3, B7-H6 is expressed on the surface of tumor cells, but not normal cells. Thus, the NKp30 receptor-NKp30 ligand system provides a relatively specific system for immune cells to recognize tumor cells. Also, B7H6 and other NKp30 ligands reportedly are expressed on infected cells, e.g., virally infected cells.

NKp30 associates with CD3ζ and FcRγ for signal transduction. There exist three isoforms of NKp30 (i.e., A, B and C), which differ in signaling capacity in NK cells (Delahaye et al., Nat Med. 2011 June; 17(6):700-7). Isoforms A and B were reported to efficiently interact with CD3ζ and are associated with good prognosis of gastrointestinal stromal tumors, whereas isoform C poorly associate with CD3ζ and linked to poor prognosis. Specifically, isoform A was demonstrated to associate with CD3ζ upon NKp30 cross-linking, whereas isoform B was demonstrated to constitutively associate with CD3ζ.

WO/2006/036445 (and its U.S. counterpart, now patented as U.S. Pat. No. 7,924,298) disclose a chimeric receptor protein comprising a C-type lectin-like natural killer cell receptor, or a protein associated therewith, fused to an immune signaling receptor having an immunoreceptor tyrosine-based activation motif for reducing or eliminating a tumor. To the N-terminus of the C-type lectin-like NK cell receptor is fused an immune signaling receptor having an immunoreceptor tyrosine-based activation motif (ITAM), (Asp/Glu)-Xaa-Xaa-Tyr*-Xaa-Xaa-(Ile/Leu)-Xaa₆₋₈-Tyr*-Xaa-Xaa-(Ile/Leu) which is involved in the activation of cellular responses via immune receptors. Similarly, when employing a protein associated with a C-type lectin-like NK cell receptor, an immune signaling receptor can be fused to the C-terminus of said protein. These patent publications additionally disclose that suitable immune signaling receptors for use in the chimeric receptor include, but are not limited to, the chain of the T-cell receptor, the eta chain which differs from the ζ chain only in its most C-terminal exon as a result of alternative splicing of the ζ mRNA, the δ, γ and ε chains of the T-cell receptor (CD3 chains) and the γ subunit of the FcR1 receptor. That publication further discloses that the immune signaling receptor may be CD3ζ. (e.g., GENBANK accession number NM_198053), or human Fcε receptor-γ chain (e.g., GENBANK accession number M33195) or the cytoplasmic domain or a splicing variant thereof. Further exemplary chimeric receptors described in these publications include a fusion between NKG2D and CD3ζ or DAP10 and CD3ζ.

U.S. Pat. Nos. 10,682,378 and 9,833,476 by Zhang et al., disclose NKp30 receptor comprising CARS, T cells engineered to express same, and methods of using these NKp30 CAR expressing T cells for reducing or ameliorating, or preventing or treating, diseases and disorders such as cancer.

However, notwithstanding the foregoing, improvements in NKp30 receptors and NKp30 receptor comprising agents e.g., fusion polypeptides, CARS, and immune cells engineered to express same are desired, particularly those which possess greater affinity to target cells and/or greater ability to kill target cells, typically tumor cells.

### SUMMARY OF THE INVENTION

The present invention relates to NK-30 variants heaving improved binding and functional properties and novel compounds or agents containing these NKp30 variants.

In some embodiments the invention provides human NKp30 variant polypeptides that bind to B7H6 with higher affinity compared to the native NKp30 receptor polypeptide but which retain its fast association and/or dissociation profile and/or elicit a different cytokine profile than native NKp30 or TZ47 and/or binds to B7H6 with higher affinity compared to the native NKp30 receptor, and/or exhibits improved in vitro or in vivo tumor cell killing relative to native NKp30 and/or (vi) binds to tumor cells expressing low levels of B7H6 better than native NKp30 or TZ47.

In some embodiments the invention provides human NKp30 variants wherein the extracellular domain of NKp30 is modified to include at least one of the following modifications in the extracellular domain: (i) G at position 26, (ii) P at position 52; (iii) W or S at position 67; (iv) A at position 70; (v) G at position 74; (vi) P at position 82; (vii) D at position 91; or (viii) G at position 104.

In some embodiments the invention provides human NKp30 variants wherein the extracellular domain of NKp30 is modified to include at least one of the following modifications: (i) A at position 70; (ii) G at position 104 or P at position 82.

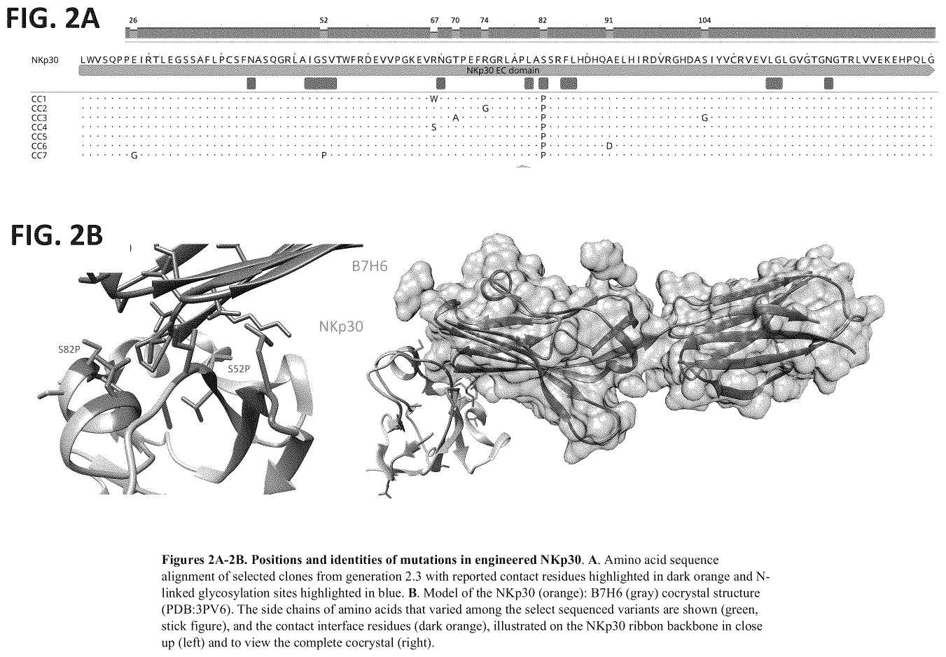

In some embodiments the invention provides human NKp30 variants wherein the extracellular domain of NKp30 is modified to comprise the same mutations as CC3 or CC5 as shown in FIG. 2.

In some embodiments the invention provides human NKp30 variants, which are directly or indirectly attached to a detectable label or effector moiety, e.g., a cytotoxin or therapeutic agent.

In some embodiments the invention provides conjugates comprising at least one variant NKp30 receptor according to any of the foregoing which are linked to one or more other protein domains, e.g., Fc domains or human CD3ζ, CD28, Dap10, CD27, and CD8, which allow for stable protein expression and/or enhanced or altered signal transduction in immune cells, optionally T or NK cells.

In some embodiments the invention provides conjugates or “antibody” drug conjugates (ADCs) or chimeric antigen receptors (CARs) comprising a human NKp30 variant according to any of the foregoing optionally comprising a drug or effector moiety, further optionally an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a radioisotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, or an imaging drug.

In some embodiments the invention provides soluble fusion proteins, optionally an Fc-fusion protein, further optionally a human IgG1, IgG2, IgG3 or IgG4 Fc fusion protein, comprising a human NKp30 variant according to any of the foregoing.

In some embodiments the invention provides a NKp30 variant, conjugate or soluble fusion protein or ADC or CAR containing according to any one of the foregoing, which comprises at least one other moiety, optionally an antibody or antibody fragment, which specifically binds to a target antigen or epitope, optionally an antigen expressed on an immune cell, further optionally a B, NK cell, Treg, T effector cell, CTL, dendritic cell, neutrophil, macrophage, monocyte, myeloid cell, eosinophil, or a precursor of any of the foregoing, further optionally wherein the antigen is CD3, PD-1, PD-L1, PD-L2, CTLA-4, CD28 or B7H6.

In some embodiments the invention provides a chimeric antigen receptor (CAR), bispecific T cell engager, or multispecific polypeptide comprising a human NKp30 variant or fusion protein or conjugate comprising according to any one of the foregoing.

In some embodiments the invention provides a chimeric antigen receptor (CAR), according to any of the foregoing which comprises signaling domains.

In some embodiments the invention provides a chimeric antigen receptor (CAR), according to the any of the foregoing, which comprises CD28 and CD3ζ intracellular domains.

In some embodiments the invention provides a chimeric antigen receptor (CAR), according to any of the foregoing which comprises: (a) NKp30 variant according to any one of the foregoing claims, (b) a transmembrane (TM) domain, and (c) an intracellular signaling (ICS) domain and optionally, a (d) a hinge that joins said NKp30 variant and said TM domain, and further optionally (e) one or more costimulatory (CS) domains.

In some embodiments the invention provides a chimeric antigen receptor (CAR), according to any of the foregoing, comprising an ICS domain derived from a cytoplasmic signaling sequence, or a functional fragment thereof, of for example, but not limited to, CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, or DAP12 or the ICS domain may be derived from a cytoplasmic signaling sequence of CD3z, or a functional fragment thereof or the hinge may be derived from CD28.

In some embodiments the invention provides a chimeric antigen receptor (CAR), according to any of the foregoing, which comprises one or more CS domains derived from a cytoplasmic signaling sequence, or functional fragment thereof, of for example, but not limited to, CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAM1, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, or CD83 ligand or a CS domain derived from a cytoplasmic signaling sequence of CD28, 4-1BB, or DAP10, or functional fragment thereof.

In some embodiments the invention provides a chimeric antigen receptor (CAR or ADC according to any of the foregoing, which comprises a cytotoxic drug directly or indirectly conjugated to the NKp30 variant.

In some embodiments the invention provides isolated polynucleotides or and/or a combination of polynucleotides vector comprising a nucleic acid or nucleic acids which separately or in combination encode a NKp30 variant polypeptide, NKp30 variant fusion protein, NKp30 variant conjugate, NKp30 variant comprising bispecific T cell engager or NKp30 variant comprising CAR or NKp30 variant comprising ADC according to any of the foregoing.

In some embodiments the invention provides an isolated polynucleotide or the combination of isolated polynucleotides according to the foregoing, which further encodes an antibody (Ab) or antigen-binding Ab fragment, which optionally may comprise a VH and a VL, optionally a monoclonal Ab, a monospecific Ab, a bispecific Ab, a multispecific Ab, a humanized Ab, a tetrameric Ab, a tetravalent Ab, a single chain Ab, a domain-specific Ab, a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a diabody, or a minibody, which antibody optionally binds to B7H6 or another antigen, optionally one expressed on an immune cell.

In some embodiments the invention provides an isolated polynucleotide or the combination of isolated polynucleotides according to any of the foregoing, which further encodes an Fc region optionally derived from the Fc region of a human IgM, a human IgD, a human IgG, a human IgE, or a human IgA, optionally of a human IgG1, a human IgG2, a human IgG3, or a human IgG4; optionally wherein the human or human-like Fc region binds to an Fc receptor (FcR), optionally an Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn).

In some embodiments the invention provides an isolated polynucleotide or combination of isolated polynucleotides according to any of the foregoing, which further encodes an (a) an AB domain that binds to a target antigen, e.g., tumor antigen or immune cell antigen; (b) a transmembrane (TM) domain; (c) an intracellular signaling (ICS) domain; (d) optionally a hinge that joins said AB domain and said TM domain; and (e) optionally one or more costimulatory (CS) domains.

In some embodiments the invention provides an isolated polynucleotide or the combination of isolated polynucleotides encoding a CAR according to any one of the foregoing, which encodes a CAR comprising at least one ICS domain derived from a cytoplasmic signaling sequence, or a functional fragment thereof, of, for example, but not limited to, CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12.

In some embodiments the invention provides an isolated polynucleotide or the combination of isolated polynucleotides encoding a CAR according to any of the foregoing, which CAR comprises at least one CS domain derived from a cytoplasmic signaling sequence, or functional fragment thereof, of, for example, but not limited to, CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAM1, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, or CD83 ligand. In certain embodiments, the CS domain may be derived from a cytoplasmic signaling sequence of CD28, 4-1BB, or DAP10, or functional fragment thereof.

In some embodiments the invention provides a vector or vectors comprising an isolated polynucleotide or combination of isolated polynucleotides according to any of the foregoing, optionally which comprises a DNA, an RNA, a plasmid, a cosmid, a viral vector, a lentiviral vector, an adenoviral vector, or a retroviral vector.

In some embodiments the invention provides a cell or cells, optionally immune cell(s), which expresses an NKp30 variant polypeptide, NKp30 variant fusion protein, NKp30 variant conjugate, NKp30 variant comprising bispecific T cell engager or NKp30 variant comprising CAR according to any of the foregoing.

In some embodiments the invention provides cell or cells according to the foregoing, which comprises a T, B, NK cell, Treg, T effector cell, CTL, dendritic cell, neutrophil, macrophage, monocyte, myeloid cell, eosinophil, or a precursor of any of the foregoing.

In some embodiments the invention provides cell or cells according to any of the foregoing, which comprises: (i) NKp30 variant polypeptide, NKp30 variant fusion protein, NKp30 variant conjugate, NKp30 variant comprising bispecific T cell engager or NKp30 variant comprising CAR according to any of the foregoing, (ii) any ADC comprising an NKp30 variant according to the foregoing, (iii) any CAR described above, (iv) any polynucleotide encoding same as described above, or (v) any vector encoding according to any of the foregoing.

In some embodiments the invention provides cell or cells according to any of the foregoing, which comprises a non-mammalian cell, optionally a plant cell, a bacterial cell, a fungal cell, a yeast cell, a protozoa cell, or an insect cell.

In some embodiments the invention provides cell or cells according to any of the foregoing, which comprises a mammalian cell, optionally a human cell, a rat cell, or a mouse cell.

In some embodiments the invention provides cell or cells according to any of the foregoing, which comprises a stem cell.

In some embodiments the invention provides cell or cells according to any of the foregoing, which comprises a primary cell, optionally a human primary cell or derived therefrom.

In some embodiments the invention provides cell or cells according to any of the foregoing, which comprises a hybridoma cell line.

In some embodiments the invention provides cell or cells according to any of the foregoing, which comprises an immune cell.

In some embodiments the invention provides cell or cells according to any of the foregoing, which is MHC+ or MHC−.

In some embodiments the invention provides cell or cells according to any of the foregoing, which comprises a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH3 cell, a TH17 cell, a TH9 cell, a TH22 cell, a follicular helper T cells, and a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, or a dendritic cell.

In some embodiments the invention provides cell or cells according to any of the foregoing, which comprises a T cell or T cell progenitor cell or NK cell.

In some embodiments the invention provides cell or cells according to any of the foregoing, which comprises a T cell which has been modified such that its endogenous T cell receptor (TCR) is (i) not expressed, (ii) not functionally expressed, or (iii) expressed at reduced levels compared to a wild-type T cell.

In some embodiments the invention provides cell or cells according to any of the foregoing, which is activated or stimulated to proliferate when the NKp30 variant containing agent, e.g., a CAR or bispecific engager or ADC binds to its target molecule.

In some embodiments the invention provides cell or cells according to any of the foregoing, which exhibit cytotoxicity against cells expressing the target molecule when the NKp30 variant containing agent, e.g., a CAR or bispecific engager or ADC binds to its target molecule.

In some embodiments the invention provides cell or cells according to any of the foregoing, which ameliorates a disease, e.g., cancer when the NKp30 variant containing agent, e.g., a CAR or bispecific engager or ADC binds to its target molecule.

In some embodiments the invention provides cell or cells according to any of the foregoing, which elicits increased expression of specific cytokines and/or chemokines when the NKp30 variant containing agent, e.g., a CAR or bispecific engager or ADC binds to its target molecule.

In some embodiments the invention provides cell or cells according to any of the foregoing, which elicits decreased expression of specific cytokines and/or chemokines when the CAR binds to its target.

In some embodiments the invention provides cell or cells according to any of the foregoing, which comprises a population of recombinant or isolated cells.

In some embodiments the invention provides a pharmaceutical composition comprising: (a) NKp30 variant according to the foregoing, (a-ii) any ADC comprising an NKp30 variant according to any of the foregoing, (iii) any CAR or bispecific T cell engager or fusion protein comprising an NKp30 variant according to the any of the foregoing, (iv) any polynucleotide or combination of polynucleotides encoding an CAR or bispecific T cell engager or fusion protein comprising an NKp30 variant according to the any of the foregoing, (v) any vector or combination of vectors encoding an NKp30 variant according to any of the foregoing, or CAR or bispecific T cell engager or fusion protein comprising an NKp30 variant according to the any of the foregoing, (vi) any cell comprising or expressing an NKp30 variant or CAR or bispecific T cell engager or fusion protein or ADC comprising an NKp30 variant according to the any of the foregoing, or (vii) any population of cells comprising any of the foregoing; and optionally (b) a pharmaceutically acceptable excipient or carrier.

In some embodiments the invention provides a composition which comprises an NKp30 variant polypeptide, NKp30 variant fusion protein, NKp30 variant conjugate, NKp30 variant comprising bispecific T cell engager or NKp30 variant comprising CAR or ADC according to any of the foregoing; a nucleic acid or vector encoding an NKp30 variant comprising bispecific T cell engager or NKp30 variant comprising CAR according to any of the foregoing; or cell which comprises or expresses an NKp30 variant polypeptide, NKp30 variant comprising fusion protein, NKp30 variant comprising conjugate, NKp30 variant comprising bispecific T cell engager or NKp30 variant comprising CAR according to any of the foregoing; and further optionally comprises a pharmaceutically acceptable carrier.

A method of treatment or prophylaxis in a subject in need thereof comprising administering a pharmaceutical composition comprising an NKp30 variant polypeptide, NKp30 variant comprising fusion protein, NKp30 variant comprising conjugate, NKp30 variant comprising bispecific T cell engager or NKp30 variant comprising CAR or ADC according to any of the foregoing; a nucleic acid or vector encoding an NKp30 variant polypeptide, NKp30 variant comprising fusion protein, NKp30 variant comprising conjugate, NKp30 variant comprising bispecific T cell engager or NKp30 variant comprising CAR or ADC according to any of the foregoing claims; or a cell, optionally an immune cell which comprises or expresses an NKp30 variant polypeptide, NKp30 variant comprising fusion protein, NKp30 variant comprising conjugate, NKp30 variant comprising bispecific T cell engager or NKp30 variant comprising CAR or ADC according to any of the foregoing; and a pharmaceutically acceptable carrier.

In some embodiments the invention provides a treatment method as above, wherein the subject has a cancer or infectious disease condition or condition associated with cells which express or overexpress one or more NKp30 ligands, optionally B7H6 or BAT3.

In some embodiments the invention provides a treatment method as above, wherein the subject has a solid tumor or hematological malignancy.

In some embodiments the invention provides a treatment method as above, which is used to treat a patient suffering from one or more of ovarian, colorectal cancer, colon and rectal cancer, lung cancer, breast cancer, brain tumor, melanoma, renal cell carcinoma, bladder cancer, leukemia, lymphoma, T cell lymphoma, multiple myeloma, gastric cancer, pancreatic cancer, uterine cervical cancer, endometrial cancer, esophageal cancer, liver cancer, head and neck squamous cell carcinoma, lung cancer, kidney cancer, bladder cancer, skin cancer, urinary tract cancer, prostate cancer, choriocarcinoma, pharyngeal cancer, laryngeal cancer, tongue cancer, oral cancer, gallbladder cancer, thyroid cancer, mesothelioma, pleural tumor, arrhenoblastoma, endometrial hyperplasia, endometriosis, embryoma, fibrosarcoma, Kaposi's sarcoma, hemangioma, cavernous hemangioma, hemangioblastoma, retinoblastoma, astrocytoma, neurofibroma, oligodendroglioma, medulloblastoma, neuroblastoma, neuroglioma, rhabdomyosarcoma, glioblastoma, osteogenic sarcoma, leiomyosarcoma, thyroid sarcoma, and Wilms tumor.

In some embodiments the invention provides a method of any one of the foregoing, which is used to treat a patient suffering from one or more of head and neck cancer, brain cancer, oral cavity cancers such as Orophyarynx cancer, Nasopharynx cancer, Hypopharynx cancer, Nasal cavity cancer, paranasal sinus cancer, Larynx cancer, Lip cancer; Lung cancers such as Non-small cell carcinoma, Small cell carcinoma, Gastrointestinal Tract cancers such as Colorectal cancer, Gastric cancer, Esophageal cancer, Anal cancer, Extrahepatic Bile Duct cancer, Cancer of the Ampulla of Vater, Gastrointestinal Stromal Tumor (GIST); Liver cancers such as Liver Cell Adenoma, Hepatocellular Carcinoma; Breast cancers, Gynecologic cancers such as Cervical cancer, Ovarian cancer, Vaginal cancer, Vulvar cancer; Gestational Trophoblastic Neoplasia, Uterine cancer, Urinary Tract cancers such as Renal cancer carcinoma, Prostate cancer, Urinary Bladder cancer, Penile cancer, Urethral cancer, Urinary Bladder cancer, Neurological Tumors such as Astrocytoma and glioblastoma, Primary CNS lymphoma, Medulloblastoma, Germ Cell tumors. Retinoblastoma, Endocrine Neoplasms. Thyroid cancer, Pancreatic cancers such as Islet Cell tumors, Insulinomas Glucagonomas, Pheochromocytoma, Adrenal carcinomas, Carcinoid tumors, Parathyroid carcinomas, Pineal gland neoplasms, Skin cancers such as Malignant melanoma, Squamous Cell carcinoma, Basal Cell carcinoma, Kaposi's Sarcoma, Bone cancers such as Osteoblastoma, Osteochondroma, Osteosarcoma; Connective Tissue neoplasms such as Chondroblastoma, Chondroma; Hematopoietic malignancies such as Non-Hodgkin Lymphoma. B-cell lymphoma, T-cell lymphoma, Undifferentiated lymphoma; Leukemias such as Chronic Myelogenous Leukemia, Hairy Cell Leukemia, Chronic Lymphocytic Leukemia, Chronic Myelomonocytic Leukemia, Acute Myelocytic Leukemia, Acute Lymphoblastic Leukemia; Myeloproliferative Disorders such as Multiple Myeloma, Essential Thrombocythemia, Myelofibrosis with Myeloid Metaplasia, Hypereosinophilic Syndrome, Chronic Eosinophilic Leukemia, Polycythemia Vera, Hodgkin Lymphoma, Childhood Cancers such as Leukemia and Lymphomas; Brain cancers, Neuroblastoma; Wilm's Tumor (nephroblastoma), Phabdomyosarcoma; Retinoblastoma; Immunotherapeutically sensitive cancers such as melanoma, kidney cancer, leukemias, lymphomas and myelomas; breast cancer; prostate cancers; colorectal cancers; cervical cancers; ovarian cancers and lung cancers.

In some embodiments the invention provides a method of preventing and/or treating cancer, or preventing cancer reoccurrence in a subject in need thereof comprising administering to said subject in need thereof an effective amount of a composition comprising an agent comprising an NKp30 variant according to any of the foregoing, e.g., an Fc fusion protein, bispecific T cell engager, CAR, ADC or other conjugate containing or a cell comprising or expressing any one of the foregoing, optionally wherein the subject has or has had at least one cancer selected from the group consisting of suffering from one or more of ovarian, colorectal cancer, colon and rectal cancer, lung cancer, breast cancer, brain tumor, melanoma, renal cell carcinoma, bladder cancer, leukemia, lymphoma, T cell lymphoma, multiple myeloma, gastric cancer, pancreatic cancer, uterine cervical cancer, endometrial cancer, esophageal cancer, liver cancer, head and neck squamous cell carcinoma, lung cancer, kidney cancer, bladder cancer, skin cancer, urinary tract cancer, prostate cancer, choriocarcinoma, pharyngeal cancer, laryngeal cancer, tongue cancer, oral cancer, gallbladder cancer, sarcoma, leukemia, melanoma, thyroid cancer, mesothelioma, pleural tumor, arrhenoblastoma, endometrial hyperplasia, endometriosis, embryoma, fibrosarcoma, Kaposi's sarcoma, hemangioma, cavernous hemangioma, hemangioblastoma, retinoblastoma, astrocytoma, neurofibroma, oligodendroglioma, medulloblastoma, neuroblastoma, neuroglioma, rhabdomyosarcoma, glioblastoma, osteogenic sarcoma, leiomyosarcoma, thyroid sarcoma, and Wilms tumor and/or optionally wherein the cancer has been determined to express or overexpress an NKp30 ligand, e.g., B7H6.

In some embodiments the invention provides a treatment method as above, which comprises administering an effective amount of a genetically modified immune cell comprising a CAR comprising an NKp30 variant according to the invention according to any of the foregoing, which is expressed on its surface, optionally a human subject.

In some embodiments the invention provides a method of producing an NKp30 variant according to any of the foregoing comprising introducing one or mutations in a NKp30 polypeptide or a nucleic acid encoding an NKp30 polypeptide and determining in one or more screens whether said NKp30 variant or the encoded NKp30 variant or a CAR, bispecific T cell engager, ADC, or fusion protein containing when expressed exhibits any combination of the following: (i) binds to B7H6 with higher affinity compared to the native receptor, (ii) retains its fast association and dissociation profile (iii) elicits a different cytokine profile than native NKp30 or TZ47, (iv) exhibits improved in vitro or in vivo tumor cell killing relative to NKp30 or TZ47 (vi) binds to tumor cells expressing low levels of B7H6 than TZ47.

In some embodiments the invention provides a method of producing an NKp30 variant as above described, wherein the resultant NKp30 variant is used to produce an NKp30 variant comprising conjugate, NKp30 variant comprising bispecific T cell engager or NKp30 variant comprising CAR according to any of the foregoing claims.

In some embodiments the invention provides a method of treating a subject with an NKp30 variant according to the invention or a CAR or bispecific T cell engager or fusion protein or ADC comprising an NKp30 variant according to any of the foregoing or a cell which expresses any of the foregoing which comprises the steps of: (a) obtaining or having obtained a biological sample, e.g., tumor biopsy, from the subject; (b) measuring the expression level of NKp30 ligands such as B7H6 in the biological sample; (c) determining whether the sample expresses or overexpresses NKp30 ligands; and (d) if the subject expresses or overexpresses NKp30 ligands, administering to the subject a therapeutically effective amount of (d-i) an NKp30 variant according to any of the foregoing or a CAR or bispecific engager, or fusion protein comprising an NKp30 variant according to any of the foregoing, (d-ii) an ADC comprising an NKp30 variant according to any of the foregoing, (d-iii) any CAR comprising an NKp30 variant according to any of the foregoing, (d-iv) any polynucleotide according to any of the foregoing, (d-v) any vector described above, (d-vi) any cell described above, (d-vii) any population of cells according to any of the foregoing, or (d-viii) any pharmaceutical composition according to any of the foregoing, optionally wherein B7H6 expression is at least 1.5 times higher than the B7H6 expression of cells of normal or healthy subjects, or is at least 1.75 times higher than the B7H6 expression of normal or healthy subjects, or is at least twice higher than the B7H6 expression of cells of normal or healthy subjects, further optionally wherein the subject is suffering from cancer, optionally pancreatic cancer, testicular cancer, cervical cancer, endometrial cancer, ovarian cancer, stomach cancer, colorectal cancer, lung cancer, mesothelioma, lymphoma, tongue cancer or other cancers previously identified.

In some embodiments the invention provides a treatment method according to any of the previous which further comprises administering a second agent, optionally an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a radioisotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, or an imaging drug.

### DETAILED DESCRIPTION OF THE FIGURES

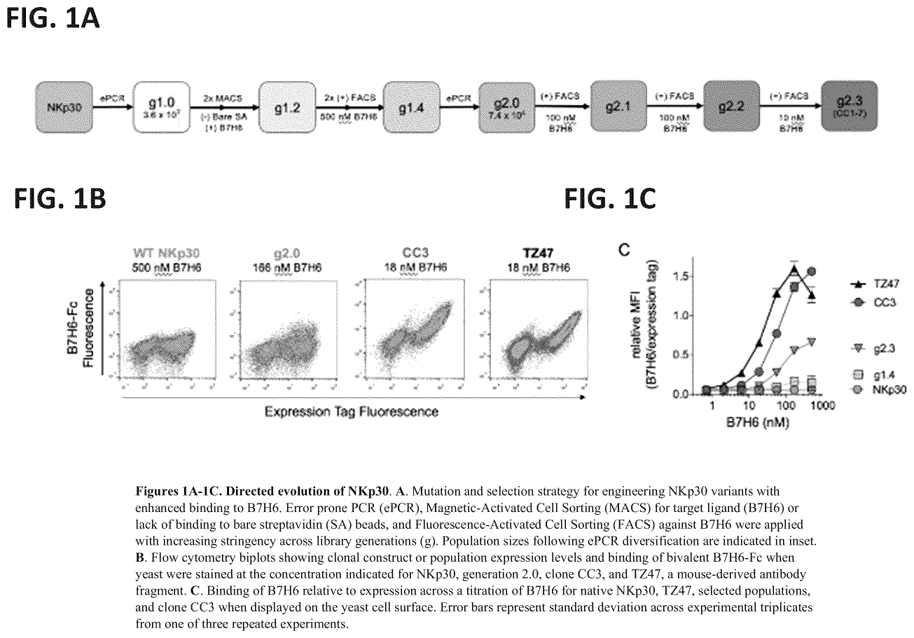

FIG. 1A-C shows the methods used for directed evolution of NKp30. A. Mutation and selection strategy for engineering NKp30 variants with enhanced binding to B7H6. Error prone PCR (ePCR), Magnetic-Activated Cell Sorting (MACS) for target ligand (B7H6) or lack of binding to bare streptavidin (SA) beads, and Fluorescence-Activated Cell Sorting (FACS) against B7H6 were applied with increasing stringency across library generations (g). Population sizes following ePCR diversification are indicated in inset. B. Flow cytometry biplots showing clonal construct or population expression levels and binding of bivalent B7H6-Fc when yeast were stained at the concentration indicated for NKp30, generation 2.0, clone CC3, and TZ47, a mouse-derived antibody fragment. C. Binding of B7H6 relative to expression across a titration of B7H6 for native NKp30, TZ47, selected populations, and clone CC3 when displayed on the yeast cell surface. Error bars represent standard deviation across experimental triplicates from one of three repeated experiments.

FIG. 2A-B shows the positions and identities of mutations in engineered NKp30. A. Amino acid sequence alignment of selected clones from generation 2.3 with reported contact residues highlighted in dark orange and N-linked glycosylation sites highlighted in blue. B. Model of the NKp30 (orange): B7H6 (gray) cocrystal structure (PDB:3PV6). The side chains of amino acids that varied among the select sequenced variants are shown (green, stick figure), and the contact interface residues (dark orange), illustrated on the NKp30 ribbon backbone in close up (left) and to view the complete cocrystal (right).

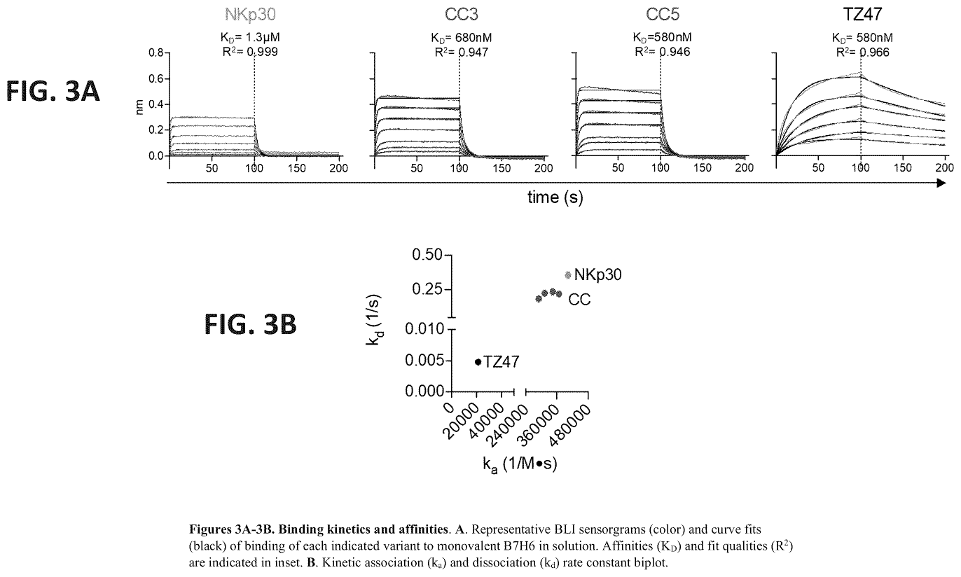

FIG. 3A-B shows binding kinetics and affinities of NKp30 variants. A. Representative BLI sensorgrams (color) and curve fits (black) of binding of each indicated variant to monovalent B7H6 in solution. Affinities (K_{D}) and fit qualities (R²) are indicated in inset. B. Kinetic association (kₐ) and dissociation (k_{d}) rate constant biplot.

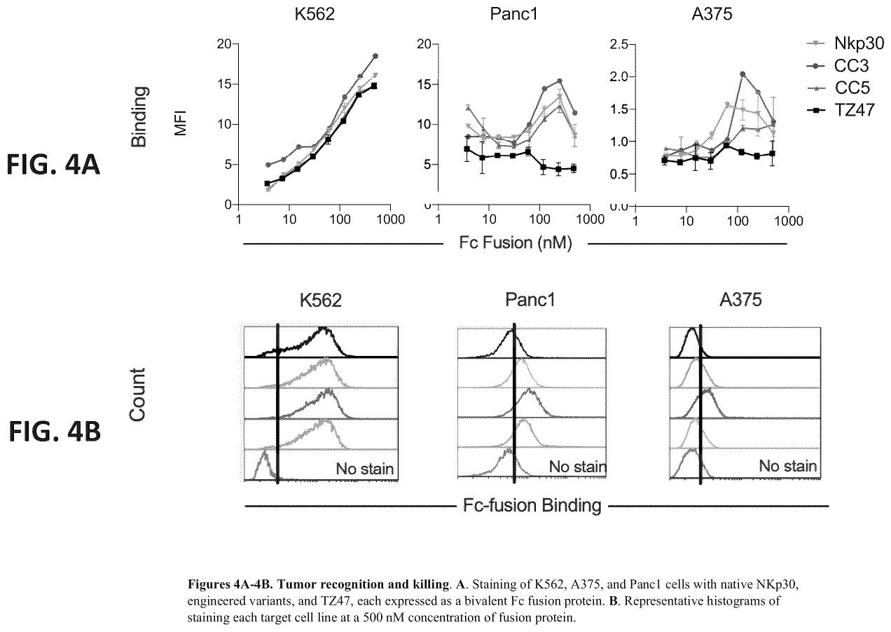

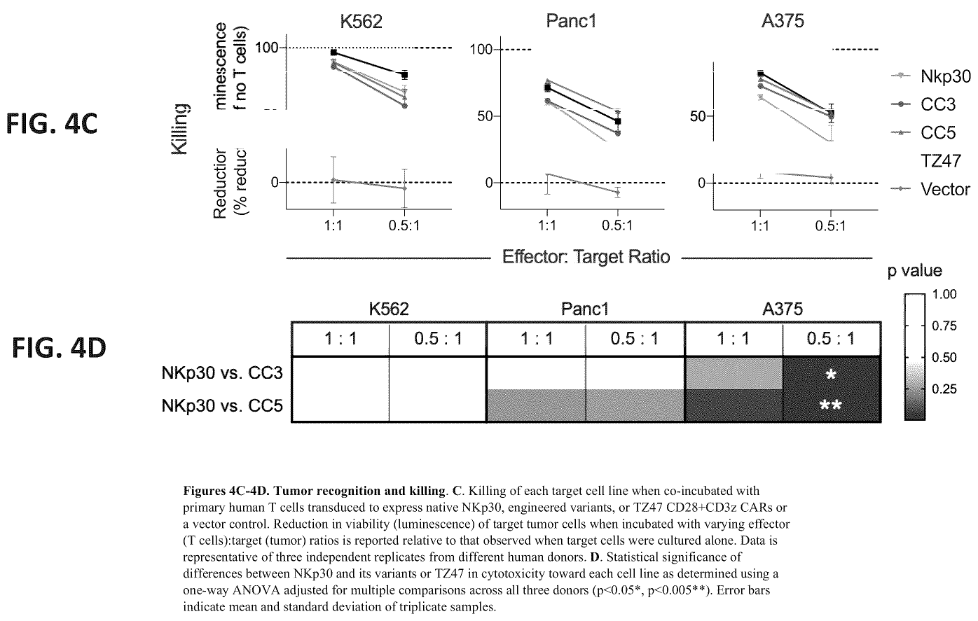

FIG. 4A-D shows tumor recognition and killing elicited by different CARs. A. Staining of K562, A375, and Panc1 cells with native NKp30, engineered variants, and TZ47, each expressed as a bivalent Fc fusion protein. B. Representative histograms of staining each target cell line at a 500 nM concentration of fusion protein. C. Killing of each target cell line when co-incubated with primary human T cells transduced to express native NKp30, engineered variants, or TZ47 CD28+CD3z CARs or a vector control. Reduction in viability (luminescence) of target tumor cells when incubated with varying effector (T cells):target (tumor) ratios is reported relative to that observed when target cells were cultured alone. Data is representative of three independent replicates from different human donors. D. Statistical significance of differences between NKp30 and its variants or TZ47 in cytotoxicity toward each cell line as determined using a one-way ANOVA adjusted for multiple comparisons across all three donors (p<0.05*, p<0.005**). Error bars indicate mean and standard deviation of triplicate samples.

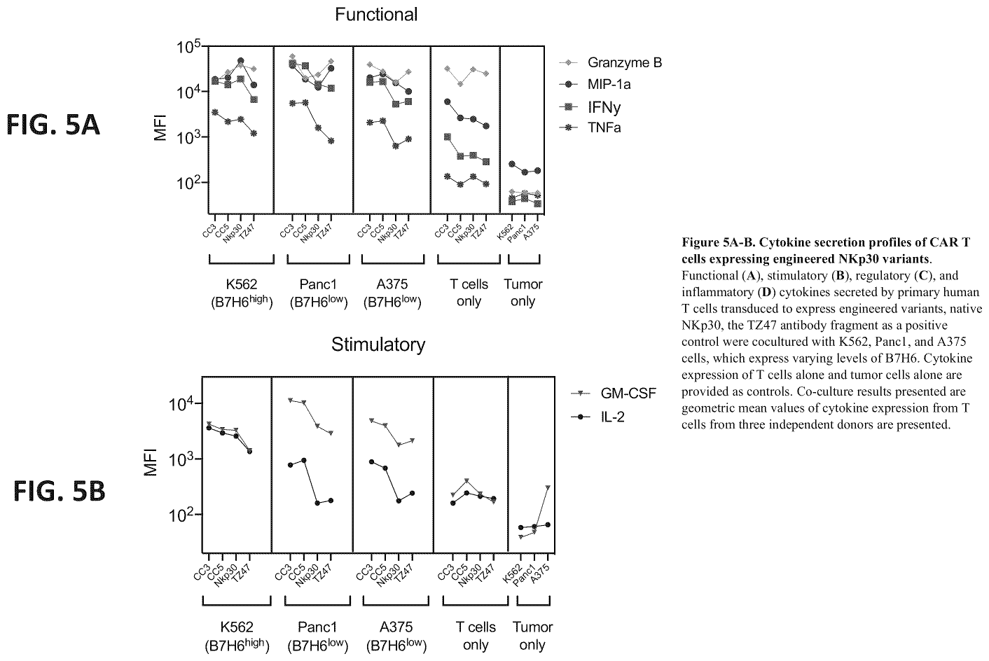

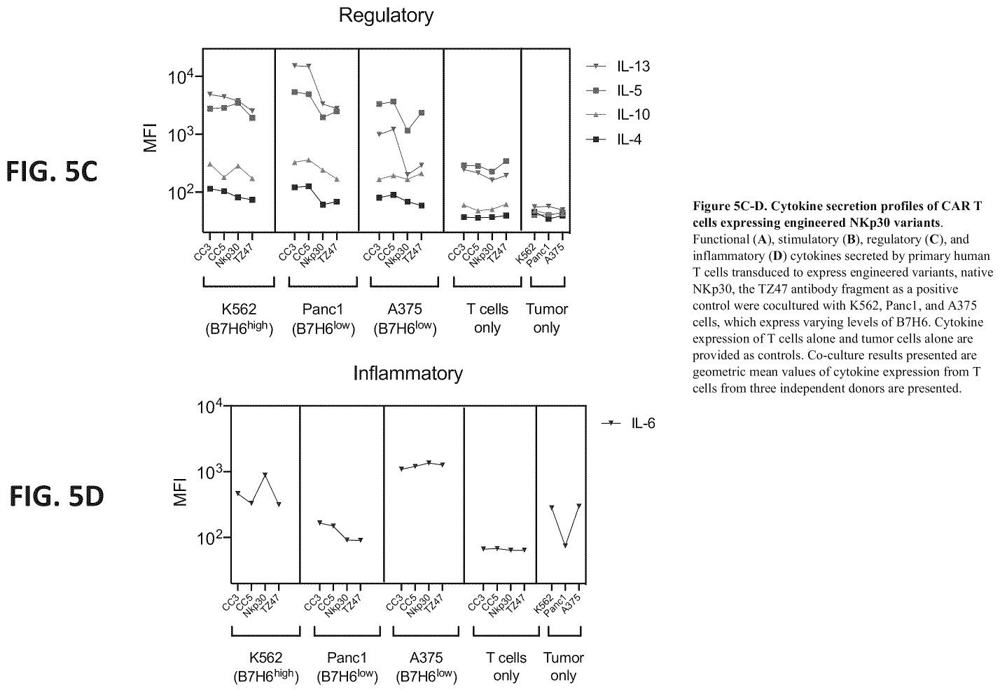

FIG. 5A-D shows the cytokine secretion profiles of CAR T cells expressing different engineered NKp30 variants. A-D. Functional (A), stimulatory (B), regulatory (C), and inflammatory (D) cytokines secreted by primary human T cells transduced to express engineered variants, native NKp30, the TZ47 antibody fragment as a positive control were cocultured with K562, Panc1, and A375 cells, which express varying levels of B7H6. Cytokine expression of T cells alone and tumor cells alone are provided as controls. Co-culture results presented are geometric mean values of cytokine expression from T cells from three independent donors are presented.

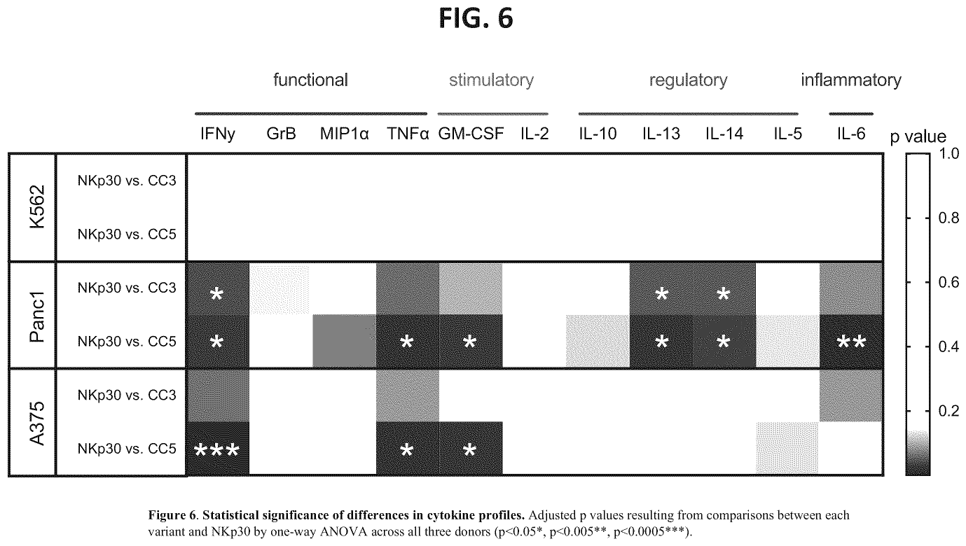

FIG. 6 shows the statistical significance of differences in cytokine profiles. Adjusted p values resulting from comparisons between each variant and NKp30 by one-way ANOVA across all three donors (p<0.05*, p<0.005**, p<0.0005***).

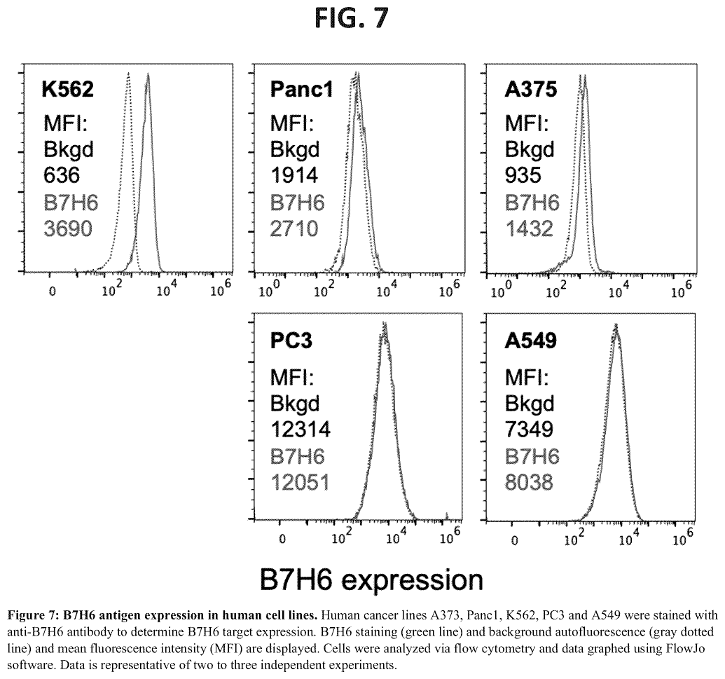

FIG. 7 shows B7H6 antigen expression in human cell lines. Human cancer lines A373, Panc1, K562, PC3 and A549 were stained with anti-B7H6 antibody to determine B7H6 target expression. B7H6 staining (green line) and background autofluorescence (gray dotted line) and mean fluorescence intensity (MFI) are displayed. Cells were analyzed via flow cytometry and data graphed using FlowJo software. Data is representative of two to three independent experiments.

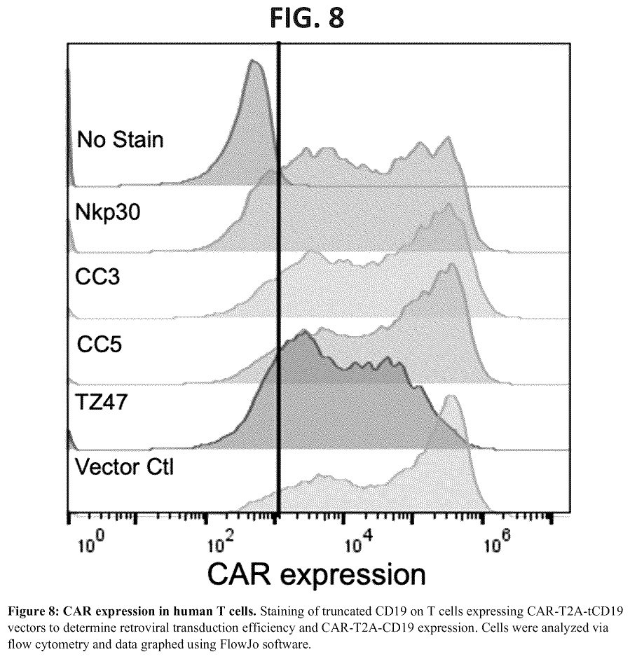

FIG. 8 shows CAR expression in human T cells. The figure shows the staining of truncated CD19 on T cells expressing CAR-T2A-tCD19 vectors to determine retroviral transduction efficiency and CAR-T2A-CD19 expression. Cells were analyzed via flow cytometry and data graphed using FlowJo software.

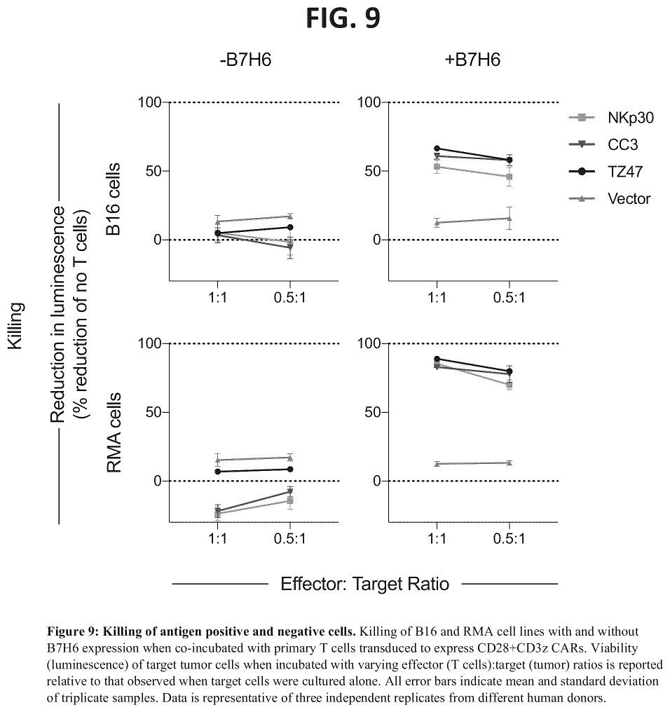

FIG. 9 shows killing of antigen positive and negative cells, particularly the killing of B16 and RMA cell lines with and without B7H6 expression when co-incubated with primary T cells transduced to express CD28+CD3z CARs. Cell viability (luminescence) of target tumor cells was assessed by incubation with varying effector (T cells):target (tumor) ratios is reported relative to that observed when target cells were cultured alone. All error bars indicate mean and standard deviation of triplicate samples. Data is representative of three independent replicates from different human donors.

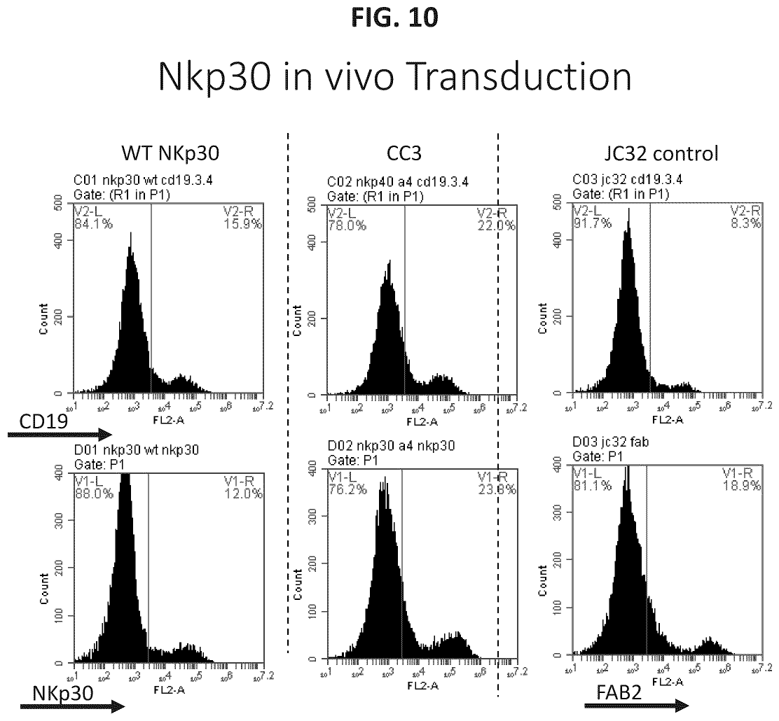

FIG. 10 shows Nkp30 in vivo transduction of CD19 and NKp30 in mice treated with WT NKp30, variant (CC3) NKp30 and JC32 control. (JC32 is Tz47 28 CAR in PFB Neo which provides a direct comparison).

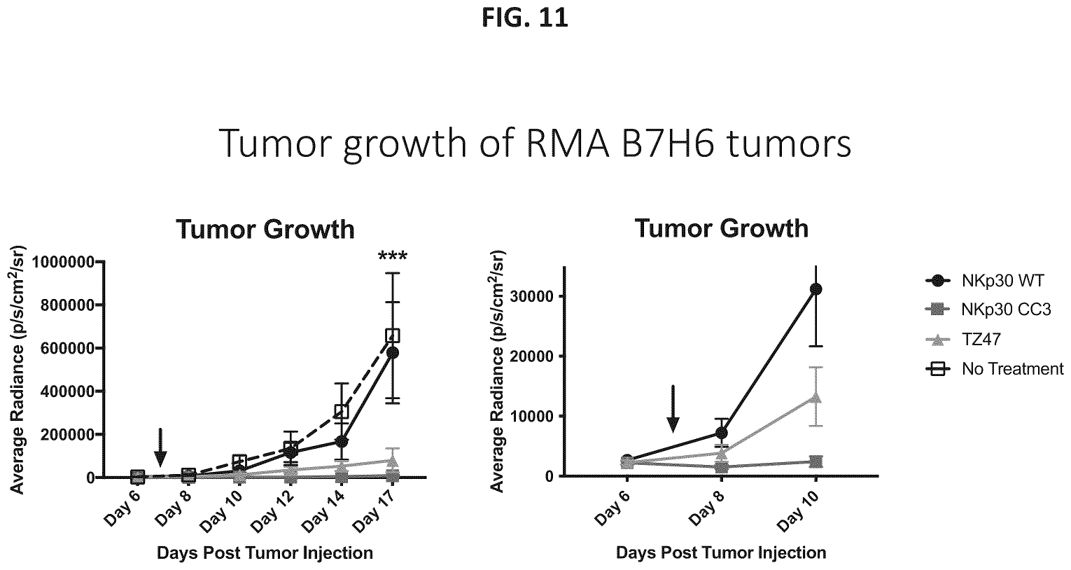

FIG. 11 compares tumor growth of RMA B7H6 tumors in NKp30 WT, NKp30 CC3 variant and TZ47 CAR treated and control (no treatment) mice. The results suggest (in view of results in FIG. 10) that the observed reduction of tumor size may be due to transduction differences.

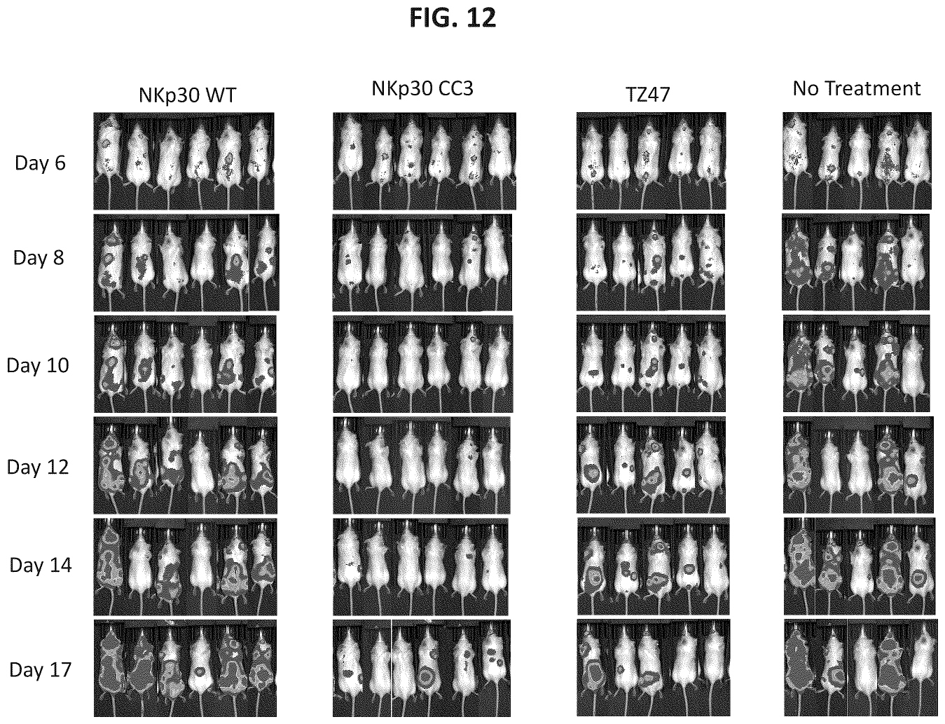

FIG. 12 contains IVIF tumor at days 6, 8, 10, 12, 14 and 17 of NKp30 WT, NKp30 CC3, and T247 CAR treated, and control (no treatment) RMA B7H6 tumor bearing mice.

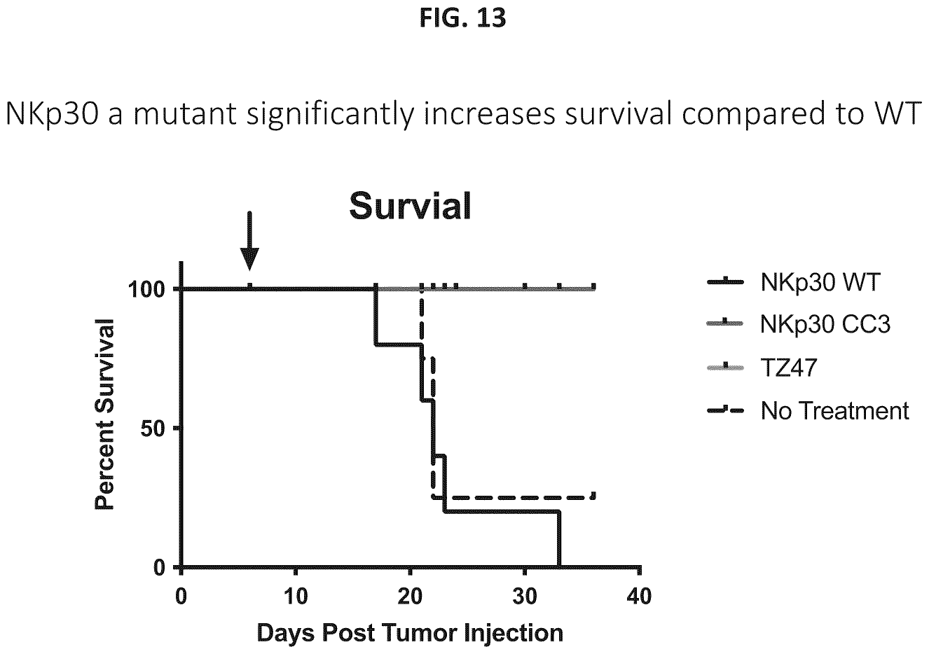

FIG. 13 contains survival data showing that RMA B7H6 tumor mice treated with the CC3 NKp30 mutant have significantly increased survival time compared to the survival time of NKp30 WT treated RMA B7H6 tumor bearing mice.

### DETAILED DESCRIPTION OF THE INVENTION

### Definitions

Prior to describing the invention in detail the following definitions are provided.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

It must also be noted that, unless the context clearly dictates otherwise, the singular forms “a,” “an,” and “the” as used herein and in the appended claims include plural reference. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

It should be understood that, unless clearly indicated otherwise, in any methods disclosed or claimed herein that comprise more than one step, the order of the steps to be performed is not restricted by the order of the steps cited.

The term “about” or “approximately” as used herein when referring to a numerical value, such as of weight, mass, volume, concentration, or time, should not be limited to the recited numerical value but rather encompasses variations of +/−10% of a given value.

The term bispecific T-cell engager generally refers to antibodies with 2 arms capable of simultaneously binding an antigen on tumor cells and a surface molecule on T cells to induce tumor lysis. An example is blinatumomab which finds use in the treatment of B cell malignancies. Herein the term will include bispecific compounds comprising an NKp30 variant and another binding moiety, e.g., an antibody or antibody fragment or antibody Fc region which binds to an immune cell, typically a T cell.

The term “allogeneic” as used herein refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “antibody” or “Ab” is used herein in the broadest sense and encompasses various antibody structures, including but not limited to full-length or full-size immunoglobulins, monoclonal antibodies, polyclonal antibodies, multispecific antibodies (e.g., bispecific antibodies), and/or antibody fragments (preferably those fragments that exhibit the desired antigen-binding activity, which is also referred to as “antigen-binding antibody fragments”). Typically, a full-size Ab comprises two pairs of chains, each pair comprising a heavy chain (HC) and a light chain (LC) interconnected by disulfide bonds. A HC typically comprises a variable region and a constant region. A LC also typically comprises a variable region and constant region. The variable region of a heavy chain (VH) typically comprises three complementarity-determining regions (CDRs), which are referred to herein as CDR 1, CDR 2, and CDR 3 (or referred to as CDR-H1, CDR-H2, CDR-H3, respectively). The constant region of a HC typically comprises a CH1 domain, a CH2 domain, and a CH3 domain. CH2 and CH3 domains form a fragment crystallizable region (Fc region), which dictates the isotype of the Ab (IgA (further divided into IgA1 and IgA2 subclasses), IgD, IgG (further divided into IgG1, IgG2, IgG3, and IgG4 subclasses), IgE, and IgM), the type of Fc receptor the Ab binds to, and therefore the effector function of the Ab. Fc receptor types include, but are not limited to, FcaR (such as FcaRI), Fca/mR, FceR (such as FceRI, FceRII), and FcgR (such as FcgRI, FcgRIIA, FcgRIIB1, FcgRIIB2, FcgRIIIA, FcgRIIIB) and their associated downstream effects are well known in the art. The variable region of a light chain (VL) also typically comprises CDRs, which are CDR 1, CDR 2, and CDR 3 (or referred to as CDR-L1, CDR-L2, CDR-L3, respectively). The constant region of a LC typically comprises a CL domain (kappa or lambda type). Antibodies can be intact immunoglobulins derived from natural sources or from recombinant sources. A molecule comprising an antibody-derived structure that enables specific binding to an antigen is referred to “antigen-binding fragment,” “AB domain,” “antigen-binding region,” or “AB region” of the Ab.

The term “antibody-drug conjugate” “Ab-drug conjugate”, or “ADC” as used herein refers to a conjugate of an NKp30 variant according to the invention and a drug. The drug may be attached to any part of the NKp30 variant via a direct or indirect attachment, such as via a linker. In some embodiments, such ADC may further comprise an antibody or antibody fragment, e.g., an Fc region.

The term “antibody fragment” or “Ab fragment” as used herein refers to any portion or fragment of an Ab, including intact or full-length Abs that may be of any class or sub-class, including IgG and sub-classes thereof, IgM, IgE, IgA and sub-classes thereof, and IgD. The term encompasses molecules constructed using one or more portions or fragments of one or more Abs. An Ab fragment can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term also encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In a specific embodiment, the antibody fragment is a scFv.

Unless otherwise stated, the term “Ab fragment” should be understood to encompass functional antibody fragments thereof. A portion of an Ab fragment that comprises a structure that enables specific binding to an antigen is referred to as “antigen-binding Ab fragment,” “AB domain,” “antigen-binding region,” or “antigen-binding region” of the Ab fragment.

A “heavy chain” or “HC” of an Ab, as used herein, refers to the larger of the two types of polypeptide chains present in all Ab molecules in their naturally occurring conformations.

A “light chain” or “LC” of an Ab, as used herein, refers to the smaller of the two types of polypeptide chains present in all Ab molecules in their naturally occurring conformations. Kappa and lambda light chains refer to the two major antibody light chain isotypes.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one genes and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated, synthesized, or can be derived from a biological sample, or might be macromolecule besides a polypeptide. Such a biological sample can include, but is not limited to a tissue sample, a cancer tissue sample, a tumor tissue sample, a leukemic cell sample, an inflamed tissue sample, and a cell or a fluid with other biological components.

The term “antigen-binding domain” or “AB domain” refers to a portion of an agent, such chimeric antigen receptor, of the present invention that allows for specific binding of the agent to another moiety such as an antigen or ligand. When the agent is an Ab, the AB domain may comprise the variable region of the Ab or a portion of the variable region, such as the CDRs. When the agent is an antigen-binding Ab fragment or an antibody-drug conjugate, the AB domain may comprise the variable region or a portion of the variable region, such as the CDRs, of the Ab that the agent is derived from. When the agent is a chimeric antigen receptor (CAR), the AB domain may be one or more extracellular domains of the CAR which have specificity for a target antigen. When the AB domain is derived from an Ab or antigen-binding Ab fragment, the AB domain may comprise the AB domain, such as the variable region or a portion of the variable region, such as the CDRs, of the Ab or antigen-binding Ab fragment that it is derived from. In some embodiments, the AB domain is an scFv.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “autologous” or “donor-derived” as used herein refers to any material derived from the same individual to whom it is later to be re-introduced.

The term “bind” refers to an attractive interaction between two molecules that results in a stable association in which the molecules are in close proximity to each other. The result of molecular binding is sometimes the formation of a molecular complex in which the attractive forces holding the components together are generally non-covalent, and thus are normally energetically weaker than covalent bonds.

The term “cancer” refers to a disease characterized by the uncontrolled growth of aberrant cells. Cancer cells can spread locally or through the bloodstream and lymphatic system to other parts of the body. Examples of various cancers relevant to the present invention include, but are not limited, pancreatic cancer, testicular cancer, cervical cancer, endometrial cancer, ovarian cancer, stomach cancer, colorectal cancer, lung cancer, mesothelioma, and tongue cancer.

The term “bispecific” as used herein refers to an entity having two binding specificities, e.g., antigen or epitopic specificities. For example a CAR or a BiTe may comprise two binding specificities, e.g., it may comprise an Nkp30 variant and therefore bind to NKp30 ligands and may further comprise an ABD such as an scFv which binds to another antigen.

The term “Chimeric Antigen Receptor” or alternatively a “CAR” refers to a set of polypeptides, typically two in the simplest embodiments, which when in an immune effector cell, provides the cell with specificity for a target cell, and with intracellular signal generation. In some embodiments, a CAR comprises at least an extracellular antigen binding domain (AB domain) or receptor, e.g., an NKp30 variant according to the invention, a transmembrane domain (TM domain) and a cytoplasmic signaling domain (also referred to herein as “an intracellular signaling domain (ICS domain)”) comprising a functional signaling domain derived from a stimulatory molecule and/or costimulatory molecule as defined below. In some embodiments, the set of polypeptides are contiguous with each other. In some embodiments, the set of polypeptides include a dimerization switch that, upon the presence of a dimerization molecule, can couple the polypeptides to one another, e.g., can couple an AB domain or receptor, e.g., NKp30 receptor polypeptide to an ICS domain. In one aspect, the stimulatory molecule is the zeta chain associated with the T cell receptor complex. In one aspect, the cytoplasmic portion of a CAR further comprises a costimulatory domain (CS domain) comprising one or more functional signaling domains derived from at least one costimulatory molecule as defined below. For example, the costimulatory molecule is chosen from the costimulatory molecules described herein, e.g., 4-1BB (i.e., CD137), DAP10 and/or CD28. In one aspect, the CAR comprises a chimeric fusion protein comprising an extracellular AB domain or an NKp30 variant according to the invention, a TM domain and an ICS domain comprising a functional signaling domain derived from a stimulatory molecule. In one aspect, the CAR comprises a chimeric fusion protein comprising an extracellular AB domain, a TM domain, an ICS domain comprising a functional signaling domain derived from a stimulatory molecule, and a CS domain comprising a functional signaling domain derived from a costimulatory molecule. In one aspect, the CAR comprises a chimeric fusion protein comprising an extracellular AB domain, a TM domain, an ICS domain comprising a functional signaling domain derived from a stimulatory molecule, and two CS domains each of the two comprising a functional signaling domain derived from a costimulatory molecule(s) that is/are same with or different from each other. In one aspect, the CAR comprises a chimeric fusion protein comprising an extracellular AB domain, a TM domain, an ICS domain comprising a functional signaling domain derived from a stimulatory molecule, and at least two CS domains each comprising a functional signaling domain derived from a costimulatory molecule(s) that is/are same with or different from each other. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The term “compete”, as used herein means that the moiety competes or blocks the binding of another moiety, e.g., it may block or inhibit the binding of an antigen or ligand with its receptor. With regard to an Ab, antigen-binding Ab fragment, of AB domain, compete means that a first Ab, antigen-binding Ab fragment, or AB domain, binds to an epitope in a manner sufficiently similar to the binding of a second Ab, antigen-binding Ab fragment, or AB domain, such that the result of binding of the first Ab, antigen-binding Ab fragment, or AB domain with its cognate epitope is detectably decreased in the presence of the second Ab, antigen-binding Ab fragment, or AB domain compared to the binding of the first Ab, antigen-binding Ab fragment, or AB domain in the absence of the second Ab, antigen-binding Ab fragment, or AB domain. The alternative, where the binding of the second Ab, antigen-binding Ab fragment, or AB domain to its epitope is also detectably decreased in the presence of the first antibody, can, but need not be the case. That is, a first Ab, antigen-binding Ab fragment, or AB domain can inhibit the binding of a second Ab, antigen-binding Ab fragment, or AB domain to its epitope without that second Ab, antigen-binding Ab fragment, or AB domain inhibiting the binding of the first Ab, antigen-binding Ab fragment, or AB domain to its respective epitope. However, where each Ab, antigen-binding Ab fragment, or AB domain detectably inhibits the binding of the other Ab, antigen-binding Ab fragment, or AB domain with its cognate epitope or ligand, whether to the same, greater, or lesser extent, the two (Ab, antigen-binding Ab fragment, or AB domain) are said to “cross-compete” with each other for binding of their respective epitope(s). Both competing and cross-competing Abs, antigen-binding Ab fragments, or AB domains are encompassed by the invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing Abs, antigen-binding Ab fragments, or AB domains are encompassed and can be useful for the methods disclosed herein.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3).

A “conjugate” herein includes any polypeptide attached to another moiety such as an NKp30 variant according to the invention attached to one or more other moieties, e.g., Fc fusion proteins.

The term “conservative amino acid substitutions” herein are as commonly used in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, lie, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a 3-branched side-chain substituted for another amino acid with a 3-branched side-chain (e.g., lie, Thr, and Val), an amino acid with an aromatic side-chain substituted for another amino acid with an aromatic side chain (e.g., His, Phe, Trp, and Tyr), etc. Non-conservative amino acid substitutions are amino acid substitutions that are not conservative amino acid substitutions.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include, but are not limited to a protein selected from the group consisting of an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8alpha, CD8beta, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R beta, IL2R gamma, IL7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and a ligand that specifically binds with CD83. In embodiments wherein a CAR comprises one or more CS domain, each CS domain comprises a functional signaling domain derived from a costimulatory molecule. In some embodiments, the encoded CS domain comprises 4-1BB, CD28, or DAP10.

The term “cytokines” as used herein refers to a broad category of small proteins that are involved in cell signaling. Generally, their release has some effect on the behavior of cells around them. Cytokines may be involved in autocrine signaling, paracrine signaling and/or endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, epithelial cells, and various stromal cells. “Chemokines” are a family of cytokines generally involved in mediating chemotaxis.

The term “cytotoxicity” generally refers to any cytocidal activity resulting from the exposure of the NKp30 variant containing agents of the invention or cells comprising the same to cells expressing said ligands which bind to said NKp30 variant, typically cancer or infected cells. This activity may be measured by known cytotoxicity assays, including IFN-γ production assays. When the target cell is a cancer or tumor cell, the term “anti-cancer cytotoxicity” or “anti-tumor cytotoxicity” may be used.

The term “cytotoxin” or “cytotoxic agent” includes any agent that is detrimental to (e.g., kills) cells. Cytotoxins can be conjugated to NKp30 variants according to the invention, e.g., using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55: 199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3: 1089-1091; Senter, P. D. and Springer, C. J. (2001) Adv. Drug Deliv. Rev. 53:247-264. Antibodies of the present invention also can be conjugated to a radioactive isotope to generate cytotoxic radiopharmaceuticals, also referred to as radioimmunoconjugates.

The phrase “disease associated with NKp30” or “NKp30-associated disease” or “NKp30 ligand associated disease” or “disease associated with cells that express NKp30 ligands” includes, includes but is not limited to, a disease associated with expression or overexpression of cells that express NKp30 ligands or condition associated with cells which express NKp30 ligands including, e.g., proliferative diseases such as a cancer or malignancy or a precancerous condition; or a non-cancer-related indication associated with cells which express or overexpress NKp30 ligands. Examples of various cancers that express NKp30 ligands are known and are disclosed herein. Also, NKp30 ligand expression is associated with some infectious disorders, particularly some viral conditions.

An “effective amount” or “an amount effective to treat” refers to a dose that is adequate to prevent or treat a disease, condition, or disorder in an individual. Amounts effective for a therapeutic or prophylactic use will depend on, for example, the stage and severity of the disease or disorder being treated, the age, weight, and general state of health of the patient, another pre-existing condition, and the judgment of the prescribing physician. The size of the dose will also be determined by the active ingredient selected, method of administration, timing and frequency of administration, the existence, nature, and extent of any adverse side effects that might accompany the administration of a particular active ingredient, and the desired physiological effect. It will be appreciated by one of skill in the art that various diseases or disorders could require prolonged treatment involving multiple administrations, perhaps using the inventive NKp30 variant containing agents, nucleic acids, vectors, cells, or compositions in each or various rounds of administration.

The terms “enteral,” “enterally,” “oral,” “orally,” “non-parenteral,” “non-parenterally,” and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal.

The term “framework” as used herein refers to the non-CDR portions of the variable region of an Ab, or in some embodiments, Antigen-binding Ab fragment or an AB domain of a CAR. “Heavy chain (HC) framework” and “VH framework” are used interchangeably herein and refer to the non-CDR portion of a HC variable region, and in general, there are four framework regions (FRs) in each full-length heavy chain variable region (FR-H1, FR-H2, FR-H3, and FR-H4). “Light chain (LC) framework” and “VL framework” are used interchangeably herein and refer to the non-CDR portion of a LC variable region, and in general, there are four FRs in each full-length light chain variable region (FR-L1, FR-L2, FR-L3, and FR-L4). In some embodiments, “human HC framework”, “human VH framework”, “human-like HC framework”, or “human-like VH framework” is at least 80%, at least 85%, at least 90%, at least 92%, at least 94%, at least 95%, at least 96%, at least 97%, at least 98%, at least 99%, or 100% identical to a human HC framework. In some embodiments, “human LC framework”, “human VL framework”, “human-like LC framework”, or “human-like VL framework” is at least 80%, at least 85%, at least 90%, at least 92%, at least 94%, at least 95%, at least 96%, at least 97%, at least 98%, at least 99%, or 100% identical to a human LC framework.

The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The term “hinge”, “spacer”, or “linker” refers to an amino acid sequence of variable length typically encoded between two or more domains or portions of a polypeptide construct to confer flexibility, improved spatial organization, proximity, etc.

As used herein, “human antibody” means an antibody having an amino acid sequence corresponding to that of an antibody produced by a human and/or which has been made using any of the techniques for making human antibodies known to those skilled in the art or disclosed herein. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “humanization” of an Ab refers to modification of an Ab of a non-human origin to increase the sequence similarity to an Ab naturally produced in humans. The term “humanized antibody” as used herein refers to Abs generated via humanization of an Ab. Generally, a humanized or engineered antibody has one or more amino acid residues from a source which is non-human, e.g., but not limited to mouse, rat, rabbit, non-human primate or another mammal. These human amino acid residues are often referred to as “import” residues, which are typically taken from an “import” variable, constant or other domain of a known human sequence. Known human Ig sequences are disclosed, e.g., www.ncbi.nlm.nih.gov/entrez/query.fcgi; www.atcc.org/phage/hdb.html, each entirely incorporated herein by reference. Such imported sequences can be used to reduce immunogenicity or reduce, enhance or modify binding, affinity, avidity, specificity, half-life, or any other suitable characteristic, as known in the art. Generally, part or all of the non-human or human CDR sequences are maintained while part or all of the non-human sequences of the framework and/or constant regions are replaced with human or other amino acids. Antibodies can also optionally be humanized with retention of high affinity for the antigen and other favorable biological properties using three-dimensional immunoglobulin models that are known to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, for example, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, each entirely incorporated herein by reference, included references cited therein.

The term “iCAR” is a chimeric antigen receptor which contains inhibitory receptor signaling domains. These domains may be based, for example, on protection D1 (PD1) or CTLA-4 (CD152). In some embodiments, the CAR expressing cells of the invention are further transduced to express an iCAR. In one aspect, this iCAR is added to restrict the CAR expressing cell's functional activity to tumor cells.

The term “immune cell” refers to a cell of hematopoietic origin functionally involved in the initiation and/or execution of innate and/or adaptive immune response.

The term “intracellular signaling domain” or “ICS domain” as used herein, refers to an intracellular portion of a molecule. The intracellular signaling domain generates a signal that promotes an immune effector function of the cell transduced with a polynucleotide comprising a CAR, e.g., a CAR T cell. Examples of immune effector function, e.g., in a CAR T cell, include cytolytic activity and helper activity, including the secretion of cytokines. ICS domains include an ICS domain of a lymphocyte receptor chain, a TCR/CD3 complex protein, an Fc receptor subunit, an IL-2 receptor subunit, CD3 zeta, FcR gamma, FcR beta, CD3 gamma, CD3 delta, CD3 epsilon, CD5, CD22, CD79a, CD79b, CD66d, CD278(ICOS), Fc epsilon RI, DAP10, or DAP12.

An “isolated” biological component (such as an isolated protein, nucleic acid, vector, or cell) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

A “leader sequence” or “LS” as used herein, also referred to as “signal peptide,” “signal sequence,” “targeting signal,” “localization signal,” “localization sequence,” “transit peptide,” or “leader peptide” in the art, is a short peptide present at the N-terminus of the majority of newly synthesized proteins that are destined towards the secretary pathway. The core of the signal peptide may contain a long stretch of hydrophobic amino acids. The signal peptide may or may not be cleaved from the mature polypeptide.

The term “mammal” refers to any mammal, including, but not limited to, mammals of the order Rodentia, such as mice, rats, and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

The term “multispecific” as used herein refers to a molecule, e.g., a CAR, bispecific T cell engager, or fusion protein having two or more binding specificities. For example an NKp30 variant containing CAR may be engineered to have multiple binding specificities.

The term “NKp30” or “Natural cytotoxicity triggering receptor 3” or “NCR3” or “CD337” (cluster of differentiation 337) refers to a receptor that is expressed on NK cells which recognizes B7-H6, which ligand is expressed on many tumors but few normal cells. NCKp30 belongs to the family of NCR membrane receptors together with NCR1 (NKp46) and NCR2 (NKp44). The gene for NKp30 is located in the MHC class III region of the human MHC locus and encodes 190 amino acid long type I transmembrane receptor which belongs to immunoglobulin super family (IgSF). NKp30 has a mass of 30 kDa and includes one Ig-like extracellular domain which is 138 amino acids long, a 19 amino acid transmembrane (TM) domain and a 33 amino acid cytoplasmic tail. The Ig-like domain consists of 2 antiparallel beta-sheets linked by a disulfide bond. The extracellular domain contains two potential sites for N-linked glycosylation involved in ligand binding. The TM domain contains a positively charged arginine residue, which associates with negatively charged aspartate in TM domain of ITAM adaptor molecules CD3ζ and FCεRIγ. This is a common feature of other NK cell activating receptors as well. Accordingly the cytoplasmic tail lacks typical ITAM consensus sequence. As used herein, NKp30 typically refers to a human NKp30 polypeptide or variant or polynucleotide encoding (or orthologs in other species if the context so indicates). Different NKp30 isoforms have been reported. The present invention unless stated otherwise includes all NKp30 isoforms.

For example human NKp30 isoform A gene has Genbank accession number NP_667341.1 having the sequence:

MAWMLLLILIMVHPGSCALWVSQPPEIRTLEGSSAFLPCSFNASQG
RLAIGSVTWFRDEVVPGKEVRNGTPEFRGRLAPLASSRFLHDHQA
ELHIRDVRGHDASIYVCRVEVLGLGVGTGNGTRLVVEKEHPQLGA
GTVLLLRAGFYAVSFLSVAVGSTVYYQGKCLTWKGPRRQLPAVVP
APLPPPCGSSAHLLPPVPGG.

NKp30 plays a major role in NK anti-tumor response and immunosurveillance, mainly by activating NK cell cytotoxicity and cytokine secretion. Direct killing happens similarly to other natural cytotoxicity receptors (NCRs) such as NKp44 and NKp46. NCR3 has a wide range of non-MHC ligands secreted or expressed by cancer or virus-infected cells, e.g. to heparan sulfate glycosaminoglycans (HS GAGs) and B7-H6. Heparan sulfate epitopes are in healthy tissue as well as on tumor cells, where HS GAGs are changed or differ in ligands (HMGB1, S100A8/A9) in contrast to healthy tissue. In addition, interaction of NCR with HS GAGs can facilitate binding to other cellular ligands. Thus via heparan sulfate epitopes NCRs can bind to the same ligands and exert similar reactions and at the same time also have their own unique interacting partners. It is also known that heparan sulfate epitopes lead to better signaling through growth factor receptors, NCRs could be thus evolved to recognize unusual HS GAGs on malignant cells as transformed cell patterns.

Ligation of NKp30 and intracellular protein HLA-B-associated transcript 3 (BAT3) released by tumour cells to extracellular matrix results in NK and dendritic cell cross-talk. Human cytomegalovirus protein pp65 is another ligand of NKp30. The ligation leads to disruption of the interaction between NKp30 and CD3ζ and thus decreases the activation of NK cells and its cytotoxicity. This is a mechanism of HMCV to evade NK cell surveillance. Patients with primary Sjögren's syndrome express higher levels of NKp30+ NK cells (and its ligation with B7-H6 expressed in salivary glands) in comparison to healthy controls.

As used herein, “NKp30 variant” typically refers to a human NKp30 polypeptide comprising one or more mutations relative to a native or endogenous NKp30 polypeptide or polynucleotide encoding such a variant. Typically a human NKp30 variant polypeptide according to the invention will bind to B7H6 with higher affinity compared to the native receptor but retain its fast association and dissociation profile and/or elicit a different cytokine profile than native NKp30 or TZ47 and/or bind to B7H6 or other NKp30 ligand with higher affinity compared to the native receptor, and/or exhibits improved in vitro or in vivo tumor cell or infected cell killing relative to the native NKp30 and/or (vi) bind to tumor cells or infected cells, e.g., virus infected cells expressing low levels of B7H6 or other NKp30 ligand better than native NKp30 or TZ47.

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The term “parenteral” or “parenterally” as used herein includes any route of administration of a compound or composition, characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

The term “pharmaceutically acceptable excipient,” “pharmaceutical excipient,” “excipient,” “pharmaceutically acceptable carrier,” “pharmaceutical carrier,” or “carrier” as used herein refers to compounds or materials conventionally used in pharmaceutical compositions during formulation and/or to permit storage.

The term “promoter”, as used herein, is defined as a DNA sequence recognized by the synthetic machinery of the cell, or introduced synthetic machinery, required to initiate the specific transcription of a polynucleotide sequence

The term “recombinant” means a polynucleotide, a protein, a cell, and so forth with semi-synthetic or synthetic origin which either does not occur in nature or is linked to another polynucleotide, a protein, a cell, and so forth in an arrangement not found in nature.

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. The linker may comprise portions of the framework sequences. In scFvs, the heavy chain variable domain (HC V, HCV, or VH) may be placed upstream of the light chain variable domain (LC V, LCV, or VL), and the two domains may optionally be linked via a linker (for example, the G4S X3 linker).

The term “signaling domain” refers to the functional portion of a protein which acts by transmitting information within the cell to regulate cellular activity via defined signaling pathways by generating second messengers or functioning as effectors by responding to such messengers.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. The signal may comprise a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as an immunoreceptor tyrosine-based activation motif or ITAM. Examples of an ITAM containing cytoplasmic signaling sequence include, but are not limited to, those derived from CD3 zeta, common FcR gamma (FCER1G), Fc gamma RIIa, FcR beta (Fc epsilon R1b), CD3 gamma, CD3 delta, CD3 epsilon, CD79a, CD79b, DAP10, and DAP12.

The term “subject” as used herein may be any living organisms, preferably a mammal. In some embodiments, the subject is a primate such as a human. In some embodiments, the primate is a monkey or an ape. The subject can be male or female and can be any suitable age, including infant, juvenile, adolescent, adult, and geriatric subjects. In some examples, the patient or subject is a validated animal model for disease and/or for assessing toxic outcomes. The subject may also be referred to as “patient” in the art. The subject may have a disease or may be healthy.

The term “synthetic Ab” or “synthetic antigen-binding Ab fragment” as used herein, refers to an Ab or antigen-binding Ab fragment which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “target” as used herein generally refers to the molecule that an NKp30 variant containing agent of the present invention specifically binds to, typically NKp30 ligands. The term also encompasses cells and tissues expressing the target molecule and also diseases that are associated with expression of the target, e.g., tumors or infected cells which express or overexpress NKp30 ligands.

The term “target cell” as used herein refers to a cell expressing the target molecule (such as B7H6 or another NKp30 ligand) on its cell surface. In some embodiments, the target cell is a cancer cell or tumor cell. In some embodiments, the target cell is an immune cell. In some embodiments, the target cell is a cell type that has a particular role in the pathology of a disease such as but not limited to cancer or infectious disease.

The term “target molecule” as used herein refers to a molecule that is targeted by the NKp30 variant containing agents of the present invention. The NKp30 variants of the present invention have improved binding affinity for cells which express NKp30 ligands, i.e., B7H6.

The term “transfected,” “transformed,” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transmembrane domain” or “TM domain”, is any three-dimensional protein structure which is thermodynamically stable in a membrane. This may be a single alpha helix, a transmembrane beta barrel, a beta-helix of gramicidin A, or any other structure. Transmembrane helices are usually about 20 amino acids in length. Typically, the transmembrane domain denotes a single transmembrane alpha helix of a transmembrane protein, also known as an integral protein.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. The type of disease or condition to be treated may be, for example, but are not limited to, cancer and cancer-associated diseases and conditions. Examples of cancer include, but are not limited to, pancreatic cancer, testicular cancer, cervical cancer, endometrial cancer, ovarian cancer, stomach cancer, colorectal cancer, lung cancer, mesothelioma, and tongue cancer. In specific embodiments, the effect of the “treatment” may be evaluated by the amelioration of at least one measurable physical parameter of a disease, resulting from the administration of one or more therapies (e.g., an NKp30 variant comprising CAR expressing cell). The parameter may be, for example, gene expression profiles, the mass of disease-affected tissues, inflammation-associated markers, cancer-associated markers, the number or frequency of disease-associated cells, tumor burden, the presence or absence of certain cytokines or chemokines or other disease-associated molecules, and may not necessarily discernible by the patient. In other embodiments “treat”, “treatment,” or “treating” may result in the inhibition of the progression of a disease, either physically by, e.g., stabilization of a discernible symptom, physiologically by, e.g., stabilization of a physical parameter, or both. In other embodiments the terms “treat”, “treatment” and “treating” refer to the reduction or stabilization of cancerous tissue or cells. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

The term “xenogeneic” or “xeno-” refers to a graft derived from an animal of a different species.

### NKp30 Variants of the Invention

As previously disclosed the invention in general provides NKp30 variants having improved binding and functional properties, e.g., enhanced binding to NKp30 ligand, particularly to B7H6 bearing cells and in some instances elicit a different effect on cytokine expression.

Many human cancer cells naturally express NKp30 ligands. To harness the NKp30 receptor-ligand interaction for cancer therapy, the inventors have created NKp30 variants by directed evolution which that bind to B7H6 with higher affinity compared to the native receptor but retain its fast association and dissociation profile.

Adoptive cell transfer is a rapidly expanding field of immunotherapy showing remarkable success in treating certain cancers. Chimeric Antigen Receptor (CAR) T cell therapy is a form of adoptive transfer that increases the frequency of tumor-reactive T cells by genetically modifying the patient's T cells to express engineered CARs that recognize a tumor antigen. Designing safe and effective CARs by achieving the proper balance of promoting tumor elimination while avoiding severe host pathology is an area of intense investigation. Traditional CARs contain an extracellular antigen-recognition domain, hinge domain, transmembrane domain, and intracellular signaling domain(s). The most commonly used antigen-recognition domain is a single chain variable fragment (scFv) that is constructed from the variable chains of the antigen-binding fragment of an antibody [1]. Natural receptors that recognize stress ligands, such as those expressed by natural killer (NK) cells, are less frequently employed as CARs [2-8]. Thus, it remains unclear to what extent scFv-based and natural receptor-based CAR recognition of tumor antigens differ in their abilities to mount effective tumor immunity. Further, while scFvs are frequently affinity matured and engineered in vitro, there is limited precedence to conducting the same type of directed evolution of a natural receptor for CAR T cell engineering.

There are unique challenges to repurposing tumor-specific scFvs as a CAR recognition domain. The elimination of stabilizing interactions between CH1 and the light chain, and the requirement for fusion of a non-native linker between the VH and VL domains often results in decreased thermal stability, which is associated with suboptimal scFv folding, expression, and solubility [9]. In addition, some studies suggest that scFv-based CARs are more prone to receptor aggregation that results in tonic signaling and poor CAR T cell persistence in vivo [10-13]. Indeed, recent work has aimed to ameliorate this phenotype by modifying the scFv framework sequence [14] and separating the VH and VL domains into separate constructs [15]. In contrast, native receptors, whose sequences have been honed over evolutionary timescales, appear to be less likely to exhibit these undesirable properties. Thus, engineering limited modifications to natural receptors may allow improved CAR activity without the increased risk of tonic signaling, instability, and potential immunogenicity of scFv-based CARs.

To begin to explore this possibility, the inventors considered a ligand of B7H6, a stress-induced ligand that is expressed in multiple cancer cell types [16, 17] and upregulated in other tissues in rare cases of autoimmunity [18, 19]. B7H6 recognition by the NK cell surface receptor NKp30 initiates NK cell-mediated cytotoxicity [20]. NKp30 was selected as a CAR extracellular-recognition domain because it has previously been demonstrated to induce IFN-γ production in NKp30-CAR T cells when incubated with B7H6-expressing cells and to promote tumor lysis both in vivo and in vitro [4]. In addition to NKp30 CARs, we generated multiple scFv-based CARs that specifically target B7H6 and mediate anti-tumor activity [21, 22]. Surprisingly, NKp30 CARs performed similarly to these scFvs in vitro, despite NKp30 having a reported affinity considerably weaker than B7H6-specific scFvs PB6 and TZ47, suggesting that natural receptor-ligand pairs may have been evolutionarily optimized for sensitive ligand recognition [21]. The extent to which natural receptors can be further improved for overall activity or selective activation of a subset of effector functions through directed evolution remains to be determined.

Given the positive association between scFv affinity to its target ligand and improved CAR function [23, 24], we sought to determine whether increasing the natural affinity of NKp30 to B7H6 through directed evolution could improve NKp30-CAR functionality. We disclose herein the generation of a new panel of NKp30 variants that bind to B7H6 with increased affinity compared to native NKp30. These variants showed distinct cytokine expression profiles against tumors expressing varying levels of B7H6. Despite more similar killing ability among NKp30-, NKp30 variant-, and TZ47 scFv-based CARs, each CAR elicited distinct cytokine profiles. Most notably, the newly engineered higher affinity NKp30 variants, in some instances, exhibited a decrease in IL-6 expression, which has been associated with cytokine release syndrome (CRS), while exhibiting retained or elevated expression of desirable cytokines.

The experimental examples disclosed herein provide proof-of-concept for fine-tuning the affinity of existing natural receptors to function more optimally in a CAR T cell setting.

Toward that end the present invention provides novel NKp30 variant polypeptides that bind to B7H6 with higher affinity compared to the native NKp30 receptor polypeptide but which retain its fast association and/or dissociation profile and/or elicit a different cytokine profile than native NKp30 or TZ47 and/or bind to B7H6 with higher affinity compared to the native NKp30 receptor, and/or exhibit improved in vitro or in vivo tumor cell killing relative to native NKp30 and/or (vi) binds to tumor cells expressing low levels of B7H6 better than native NKp30 or TZ47.

In some exemplary embodiments the invention provides human NKp30 variants wherein the extracellular domain of NKp30 is modified to include at least one of the following modifications in the extracellular domain: (i) G at position 26, (ii) P at position 52; (iii) W or S at position 67; (iv) A at position 70; (v) G at position 74; (vi) P at position 82; (vii) D at position 91; or (viii) G at position 104.

In some exemplary embodiments the invention provides human NKp30 variant wherein the extracellular domain of NKp30 is modified to include at least one of the following modifications: (i) A at position 70; (ii) G at position 104 or P at position 82.

In preferred exemplary embodiments the invention provides human NKp30 variants wherein the extracellular domain comprises the same or additional mutations as CC3 or CC5 as shown in FIG. 2.

In general these human NKp30 variant will be used as diagnostic and/or as therapeutic agents for detecting and/or treating conditions wherein the disease pathology involves cells which express or overexpress NKp30 ligands, e.g., B7H6 and BAT3. Typically the NKp30 variants will be used to produce fusion proteins, and conjugates such as bispecific T cell engagers or chimeric antigen receptors or ADCs and cells which express same which are used as therapeutic agents, typically for treating tumors where the disease pathology involves B7H6 expressing cells, e.g., B7H6 expressing tumor cells.

When the subject NKp30 variants are used for detection they will be bound to a moiety that provides for NKp30 variant/B7H6 complexes to be detected upon the binding of the NKp30 ligand expressing cells, e.g., tumor or virally infected cells. The detectable label may e.g., comprise a fluorophore, radionuclide, enzyme and the like.

When the subject NKp30 variants are used for therapy they will be directly or indirectly attached or conjugated to a moiety which provides for therapeutic efficacy. In some instances the NKp30 variant may be bound to an effector moiety, e.g., a cytotoxin or therapeutic agent which elicits activity, generally killing when the resultant conjugate binds to a target cell, e.g., a tumor or virally infected cell which expresses B7H6 or BAT3 ligands.

In some instances when the subject NKp30 variants are used for therapy they will be directly or indirectly attached or conjugated to produce a conjugate comprising at least one variant NKp30 receptor which is linked to one or more other protein domains, e.g., Fc domains or signaling domains such as human CD3ζ, CD28, Dap10, CD27, and CD8, which allow for stable protein expression and/or enhanced or altered signal transduction in immune cells, optionally T or NK cells. In exemplary embodiments the protein domain may comprise an Ig Fc region, typically a human Ig Fc region, e.g., a human IgG1, IgG2, IgG3 or IgG4 Fc region which optionally may be mutated to enhance or inhibit a desired effector function, e.g., ADCC, complement activity, FcR binding, phagocytosis, et al.

In some instances when the subject NKp30 variants are used for therapy they will comprise a conjugate or “antibody” drug conjugate (ADC) or chimeric antigen receptor (CAR) comprising a human NKp30 variant as provided herein optionally comprising a drug or effector moiety. Such drug or effector moiety may optionally comprise an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a radioisotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, or an imaging drug.

In some instances when the subject NKp30 variants are used for therapy they will comprise soluble fusion proteins, optionally an Fc-fusion protein, further optionally a human IgG1, IgG2, IgG3 or IgG4 Fc fusion protein, comprising a human NKp30 variant as disclosed herein.

In some instances when the subject NKp30 variants are used for therapy they will comprise conjugates or soluble fusion proteins or ADCs or CARs, which comprises at least one other moiety, optionally an antibody or antibody fragment, which specifically binds to a target antigen or epitope, optionally an antigen expressed on an immune cell, further optionally a B, NK cell, Treg, T effector cell, CTL, dendritic cell, neutrophil, macrophage, monocyte, myeloid cell, eosinophil, or a precursor of any of the foregoing, further optionally wherein the antigen is CD3, PD-1, PD-L1, PD-L2, CTLA-4, CD28 or B7H6.

In some instances when the subject NKp30 variants are used for therapy it will be in the form of a chimeric antigen receptor (CAR) comprising a human NKp30 variant or fusion protein or conjugate comprising signaling domains, e.g., CD3ζ signaling domains.

In some instances when the subject NKp30 variants are used for therapy it will be in the form of a chimeric antigen receptor (CAR) comprising: (a) NKp30 variant according to any one of the foregoing claims, (b) a transmembrane (TM) domain, and (c) an intracellular signaling (ICS) domain and optionally, a (d) a hinge that joins said NKp30 variant and said TM domain, and further optionally (e) one or more costimulatory (CS) domains.

In some instances when the subject NKp30 variants are used for therapy it will be in the form of a fusion protein comprising a human or human-like Fc region may be at least 80%, at least 85%, at least 90%, at least 92%, at least 94%, at least 95%, at least 96%, at least 97%, at least 98%, at least 99%, or 100% identical to a human Fc region, optionally human or human-like Fc region may be derived from a human IgM, IgD, IgG, IgE, or IgA, preferably IgG1, IgG2, IgG3, or IgG4.

In certain embodiments, when the subject NKp30 variants are used for therapy it will be in the form of a fusion protein comprising a human or human-like Fc region the human-like Fc region may bind to an Fc receptor (FcR). The FcR may be, but is not limited to, Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn).

In some embodiments when the subject NKp30 variants are used for therapy it will be in the form of a fusion protein comprising a human or human-like Fc region the Fc region may comprise one or more modifications. Certain amino acid modifications in the Fc region are known to modulate effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9(1): 63-73; Dall'Acqua W. F. et al., J Biol Chem. 2006 Aug. 18; 281(33):23514-24. Epub 2006 Jun. 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289(6): 3571-3590).

When the Fc is an IgG1 Fc, the Fc region may comprise one or more amino acid substitutions. The substitution may be, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, 1332E, S298A, E333A, K334A, L234Y, L235Q G236W, S239M, H268D, D270E, K326D, A330M, K334E, G236A, K326W, S239D, E333S, S267E, H268F, S324T, E345R, E430G, S440Y M428L, N434S, L328F, M252Y, S254T, T256E, and/or any combination thereof (the residue numbering is according to EU or Kabat numbering) (Dall'Acqua W. F. et al., J Biol Chem. 2006 Aug. 18; 281(33):23514-24. Epub 2006 Jun. 21; Wang X. et al., Protein Cell. 2018 January; 9(1): 63-73). The Fc region may further comprise one or more additional amino acid substitutions. The substitution may be, for example, but is not limited to, A330L, L234F, L235E, P3318, and/or any combination thereof (the residue numbering is according to EU or Kabat numbering).

When the Fc is an IgG2 Fc, the Fc region may comprise one or more amino acid substitutions. The substitution may be, for example, but is not limited to, P238S, V234A, G237A, H268A, H268Q, H268E, V309L, N297A, N297Q A330S, P331S, C232S, C233S, M252Y, S254T, T256E, and/or any combination thereof (the residue numbering is according to EU or Kabat numbering). The Fc region may further comprise one or more additional amino acid substitutions. The substitution may be, for example, but is not limited to, M252Y, S254T, T256E, and/or any combination thereof (the residue numbering is according to EU or Kabat numbering).

When the Fc is an IgG3 Fc, the Fc region may comprise one or more amino acid substitutions. The substitution may be, for example, but is not limited to, E235Y (the residue numbering is according to EU or Kabat numbering).

When the Ab is an IgG4, the Fc region may comprise one or more amino acid substitutions. The substitution may be, for example, but is not limited to, E233P, F234V, L235A, G237A, E318A, S228P, L236E, S241P, L248E, T394D, M252Y, S254T, T256E, N297A, N297Q, and/or any combination thereof (the residue numbering is according to EU or Kabat numbering). The substitution may be, for example, S228P (the residue numbering is according to EU or Kabat numbering).

In some embodiments, the glycan of the human-like Fc region may be engineered to modify the effector function (for example, see Li T. et al., Proc Natl Acad Sci USA. 2017 Mar. 28; 114(13):3485-3490. doi: 10.1073/pnas.1702173114. Epub 2017 Mar. 13).

In some embodiments, the NKp30 variant agent of the present invention may be an “antibody-drug conjugate” or (ADC). The ADC may comprise: (a) any NKp30 variant according to the invention as described herein; and (b) a drug directly or indirectly conjugated to the NKp30 variant.

In some embodiments, the drug may be, but not limited to, an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a radioisotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, and an imaging drug.

The toxin may be a bacterial, fungal, plant, or animal toxin, or a fragment thereof. Examples include, but are not limited to, diphtheria A chain, diphtheria toxin, exotoxin A chain, ricin A chain, abrin A chain, modeccin A chain, alpha sarcin, Aleurites fordii protein, a dianthin protein, or a Phytolacca Americana protein.

The anti-cancer or anti-proliferative drug may be, for example, but is not limited to, doxorubicin, daunorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, epothilone, or a derivative thereof (for example, see Polakis P. et al., Pharmacol Rev. 2016 January; 68(1):3-19. doi: 10.1124/pr.114.009373) (the drugs may be obtained from many vendors, including Creative Biolabs®).

The radioisotope may be for example, but is not limited to, At211, I131, In131, I125, Y90, Re186, Re188, Sm153, Bi212, P32, Pb212 and radioactive isotopes of Lu.

In certain embodiments, the drug may be, but is not limited to, MMAE or MMAF.

In some embodiments, the NKp30 variant is directly conjugated to the drug to form an ADC.

In some embodiments, the NKp30 variant is indirectly conjugated to the drug to form an ADC.

Any appropriate conjugation method may be used to generate an ADC (for example, Nolting B. Methods Mol Biol. 2013; 1045:71-100. doi: 10.1007/978-1-62703-541-5_5; Jain N. et al., Pharm Res. 2015 November; 32(11):3526-40. doi: 10.1007/s11095-015-1657-7. Epub 2015 Mar. 11; Tsuchikama K. et al., Protein Cell. 2018 January; 9(1):33-46. doi: 10.1007/s13238-016-0323-0. Epub 2016 Oct. 14; Polakis P. et al., Pharmacol Rev. 2016 January; 68(1):3-19. doi: 10.1124/pr.114.009373). Examples of methods that may be used to perform conjugation include, but are not limited to, chemical conjugation and enzymatic conjugation.

Chemical conjugation may utilize, for example, but is not limited to, lysine amide coupling, cysteine coupling, and/or non-natural amino acid incorporation by genetic engineering. Enzymatic conjugation may utilize, for example, but is not limited to, transpeptidation using sortase, transpeptidation using microbial transglutaminase, and/or N-Glycan engineering.

In certain embodiments, one or more of cleavable linkers may be used for conjugation. The cleavable linker may enable cleavage of the drug upon responding to, for example, but not limited to, an environmental difference between the extracellular and intracellular environments (pH, redox potential, etc.) or by specific lysosomal enzymes.

Examples of the cleavable linker include, but are not limited to, hydrazone linkers, peptide linkers including cathepsin B-responsive linkers, such as valin-citrulline (vc) linker, disulfide linkers such as N-succinimidyl-4-(2-pyridyldithio) (SPP) linker or N-succinimidyl-4-(2-pyridyldithio)butanoate (SPDB) linker, and pyrophosphate diester linkers.

Alternatively or simultaneously, one or more of non-cleavable linkers may be used. Examples of non-cleavable linkers include thioether linkers, such as N-succinimidyl 4-(N-maleimidomethyl) cyclohexane-1-carboxylate (SMCC), and maleimidocaproyl (mc) linkers. Generally, non-cleavable linkers are more resistant to proteolytic degradation.

In some embodiments, an NKp30 variant comprising agent according to the present disclosure may be a chimeric antigen receptor (CAR). In particular, the CARs may comprise an NKp30 variant according to the invention that binds B7H6 ligands, a transmembrane (TM) domain, and an intracellular signaling (ICS) domain and may optionally comprise a hinge that joins the an NKp30 variant and said TM domain. The CAR may optionally comprise one or more costimulatory (CS) domains.

### Vectors

The present invention also provides vectors in which a polynucleotide encoding an NKp30 variant or conjugate containing such as a CAR or bispecific T cell engager or ADC of the present invention is inserted.

The vector may be, for example, a DNA vector or a RNA vector. The vector may be, for example, but not limited to, a plasmid, a cosmid, a viral replicon, or a viral vector. The viral vector may be a vector of a DNA virus, which may be an adenovirus, or an RNA virus, which may be a retrovirus. Types of vectors suite for Abs, antigen-binding Ab fragments, and/or CARs are well known in the art (for example, see Rita Costa A. et al., Eur J Pharm Biopharm. 2010 February; 74(2):127-38. doi: 10.1016/j.ejpb.2009.10.002. Epub 2009 Oct. 22; Frenzel A. et al. Front Immunol. 2013; 4: 217. Published online 2013 Jul. 29. doi: 10.3389/fimmu.2013.00217).

When the host cells are insect cells, such as for producing Abs or antigen-binding Ab fragments, insect-specific viruses may be used. Examples of the insect-specific viruses include, but are not limited to, the family of Baculoviridae, particularly the Autographa californica nuclear polyhedrosis virus(AcNPV). When the host cells are plant cells, plant-specific viruses and bacteria, such as Agrobacterium tumefaciens, may be used.

For expressing vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of nucleic acids encoding an NKp30 variant containing agents is typically achieved by operably linking a nucleic acid encoding the an NKp30 variant polypeptide or conjugate containing such as an Fc fusion protein, CAR, BiTe, to a promoter, and incorporating the construct into an expression vector. The vectors can be suitable for replication and integration eukaryotes. Typical cloning vectors contain transcription and translation terminators, initiation sequences, and promoters useful for regulation of the expression of the desired polynucleotide.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, γ-retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Various promoter sequences may be used, including, but not limited to the immediate early cytomegalovirus (CMV) promoter, the CMV-actin-globin hybrid (CAG) promotor, Elongation Growth Factor-1α (EF-1α), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

In some embodiments, the selectable marker gene comprises a polynucleotide encoding truncated CD19 (trCD19). When a marker such as trCD19, which can be expressed on the cell surface is used, the expression of the marker may be determined via any available technique including, but not limited to, flow cytometry or immunofluorescence assays. Expression of such a marker typically indicates successful introduction and expression of the transgene(s) introduced together with the marker gene. Therefore, cells expressing the an NKp30 variant containing agent of the invention may be, for example, selected based on the expression of the marker.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Transfection/Transduction

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20 degrees Celsius. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., “1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Cells

Also provided are cells, cell populations, and compositions containing the cells, e.g., cells comprising a polynucleotide encoding an NKp30 variant or agent containing, e.g., an Fc fusion protein, CAR, bispecific T cell engager, ADC containing an NKp30 variant according to the invention. Cells expressing an NKp30 variants or conjugates containing may be administered to a subject or may be incorporated in a composition to be administered to a subject. Among the compositions are pharmaceutical compositions and formulations for administration, such as for adoptive cell therapy.

Also provided are therapeutic methods for administering the NKp30 variants or conjugates containing or the cells which express the NKp30 variants or conjugates containing and compositions containing any of the foregoing to subjects, e.g., patients.

### Cell Types

Thus, also provided are cells expressing the NKp30 variants or conjugates containing of the present invention.

For expressing an NKp30 variants or conjugates containing, any appropriate cells may be used. For example, cells may be: (i) prokaryotic cells, such as gram-negative bacteria and gram-positive bacteria; or (ii) eukaryotic cells, such as yeast, filamentous fungi, protozoa, insect cells, plant cells, and mammalian cells (reviewed in Frenzel A. et al. Front Immunol. 2013; 4: 217. Published online 2013 Jul. 29. doi: 10.3389/fimmu.2013.00217).

Specific examples of gram-negative bacteria that are suited for production of the NKp30 variants or conjugates containing of the present invention include, but are not limited to, E. coli, Proteus mirabilis, and Pseudomonas putidas. Specific examples of gram-positive bacteria include, but are not limited to, Bacillus brevis, Bacillus subtilis, Bacillus megaterium, Lacto-bacilluszeae/casei, and Lactobacillus paracasei. Specific examples of yeast bacteria that are suited for production of the NKp30 variants or conjugates containing of the present invention include, but are not limited to, Pichia pastoris, Saccharomyces cerevisiae, Hansenula polymorpha, Schizosaccharomyces pombe, Schwanniomyces occidentalis, Kluyveromyces lactis, and Yarrowia lipolytica. Specific examples of filamentous fungi that are suited for production of the NKp30 variants or conjugates containing of the present invention include, but are not limited to, the genera Trichoderma and Aspergillus, A. niger (subgenus A. awamori), Aspergillus oryzae, and Chrysosporium lucknowense. Specific examples of protozoa that are suited for production of the NKp30 variants or conjugates containing of the present invention include, but are not limited to, Leishmania tarentolae. Specific examples of insect cells that are suited for production of the NKp30 variants or conjugates containing of the present invention include, but are not limited to, insect cell lines like Sf-9 and Sf-21 of Spodoptera frugiperda, DS2 cells of Drosophila melanogaster, High Five cells (BTI-TN-5B1-4) of Trichopulsia ni, or Schneider2 (S2) cells of D. melanogaster. They can be efficiently transfected with insect-specific viruses from the family of Baculoviridae, particularly the Autographa californica nuclear polyhedrosis virus (AcNPV). Specific examples of mammalian cells that are suited for production of the NKp30 variants or conjugates containing of the present invention include, but are not limited to, Chinese hamster ovary (CHO) cells, the human embryonic retinal cell line Per.C6 [Crucell, Leiden, Netherlands], CHO-derived cell lines such as K1-, DukXB11-, Lec13, and DG44-cell lines, mouse myeloma cells such as SP 2/0, YB 2/0, and NSO cells, GS-NSO, hybridoma cells, baby hamster kidney (BHK) cells, and the human embryonic kidney cell line HEK293, HEK293T, HEK293E, and human neuronal precursor cell line AGE1.HN (Probiogen, Berlin, Germany).

Alternatively, genetically modified organisms such as transgenic plants and transgenic animals may be used. Exemplary plants that may be used include, but are not limited to, tobacco, maize, duckweed, Chlamydomonas reinhardtii, Nicotiana tabacum, Nicotianaben thamiana, and Nicotiana benthamiana. Exemplary animals that may be used include, but are not limited to mouse, rat, and chicken.

For expressing an NKp30 variant containing CAR, the cells generally are eukaryotic cells, such as mammalian cells, and typically are human cells, more typically primary human cells, e.g., allogeneic or autologous donor cells. The cells for introduction of the CAR may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immunity, e.g., myeloid cells, including monocytes, macrophages, dendritic cells, neutrophils, eosinophils, basophils, or mast cells, or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Alternatively, an immortalized cell or a cell line may be used for expressing a CAR of the present disclosure. Such examples include, but are not limited to, a T cell line, a CD4+ T cell line, a CD8+ T cell line, a regulatory T cell line, an NK-T cell line, an NK cell line (e.g., NK-92), a monocyte line, a macrophage line, a dendritic cell line, and a mast cell line. Furthermore, a desired cell type for CAR expression, for example T cells or NK cells may be generated from a stem cell, such as an embryonic stem cell, iPSCs, or hematopoietic stem cell.

With reference to the subject to be treated with cells expressing an NKp30 variant comprising CAR, the cells may be allogeneic and/or autologous. Among the methods include off-the-shelf methods. In some embodiments, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

In some embodiments, the cells are T cells. Among the sub-types and subpopulations of T cells and/or of CD4⁺ and/or of CD8⁺ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, α/β T cells, and δ/γ T cells.

In some embodiments, the cells are natural killer (NK) cells, Natural Killer T (NKT) cells, cytokine-induced killer (CIK) cells, tumor-infiltrating lymphocytes (TIL), lymphokine-activated killer (LAK) cells, or the like. In some embodiments, the cells are monocytes or granulocytes, e.g., myeloid cells, macrophages, neutrophils, dendritic cells, mast cells, eosinophils, and/or basophils. CAR-expressing phagocytic cells expressing may be able to bind to and phagocytose or nibble target cells (Morrissey M. A. et al., Elife. 2018 Jun. 4; 7. pii: e36688. doi: 10.7554/eLife.36688).

In some embodiments, the cells are derived from cell lines, e.g., T cell lines. The cells in some embodiments are obtained from a xenogeneic source, for example, from mouse, rat, non-human primate, and pig.

### Therapeutic Applications

NKp30 variants and conjugates containing such as bispecific T cell engagers, Ig fusion proteins, ADCs or CARS, nucleic acids encoding such NKp30 variants and conjugates containing, vectors encoding such NKp30 variants and conjugates containing, isolated cells obtained by the methods described above, or cell lines derived from such isolated cells, and/or pharmaceutical compositions comprising thereof can be used as a medicament in the treatment of a disease, disorder, or condition in a subject. In some embodiments, such a medicament can be used for treating a disease associated with NKp30 ligand expressing cells such as cancer or infection.

### Target Diseases and Conditions

### Cancer

The NKp30 ligand-associated condition may be, for example, but not limited to, cancer and cancer-associated diseases and conditions.

In particular embodiments, the NKp30 variants and conjugates containing of the present invention may be used to treat a cancer. Nkp30 ligands such as B7H6 are upregulated in a variety of cancers, such as, but not limited to ovarian cancer, colorectal cancer, colon and rectal cancer, lung cancer, breast cancer, brain tumor, melanoma, renal cell carcinoma, bladder cancer, leukemia, lymphoma, T cell lymphoma, multiple myeloma, gastric cancer, pancreatic cancer, uterine cervical cancer, endometrial cancer, esophageal cancer, liver cancer, head and neck squamous cell carcinoma, lung cancer, kidney cancer, bladder cancer, skin cancer, urinary tract cancer, prostate cancer, choriocarcinoma, pharyngeal cancer, laryngeal cancer, tongue cancer, oral cancer, gallbladder cancer, sarcoma, leukemia, melanoma, thyroid cancer, mesothelioma, pleural tumor, arrhenoblastoma, endometrial hyperplasia, endometriosis, embryoma, fibrosarcoma, Kaposi's sarcoma, hemangioma, cavernous hemangioma, hemangioblastoma, retinoblastoma, astrocytoma, neurofibroma, oligodendroglioma, medulloblastoma, neuroblastoma, neuroglioma, rhabdomyosarcoma, glioblastoma, osteogenic sarcoma, leiomyosarcoma, thyroid sarcoma, and Wilms tumor.

Therefore, preferred target diseases include these cancers. In some embodiments, ovarian cancer is a preferred target disease. In some embodiments, lymphoma is a preferred target disease. The NKp30 variants and conjugates containing according to the present invention may also be used to treat any disease in which B7H6 is upregulated or has a pathological role.

This therapeutic application of the inventive NKp30 variants and conjugates containing may be confirmed in suitable in vitro or in vivo disease models.

### Subject

The subject referred to herein may be any living subject. In a preferred embodiment, the subject is a mammal. The mammal referred to herein can be any mammal. As used herein, the term “mammal” refers to any mammal, including, but not limited to, mammals of the order Rodentia, such as mice and hamsters, and mammals of the order Lagomorpha, such as rabbits. The mammals may be from the order Carnivora, including Felines (cats) and Canines (dogs). The mammals may be from the order Artiodactyla, including bovines (cows) and swine (pigs) or of the order Perssodactyla, including Equines (horses). The mammals may be of the order Primates, Ceboids, or Simoids (monkeys) or of the order Anthropoids (humans and apes).

In some embodiments, the subject, to whom the NKp30 variants and conjugates containing, e.g., bispecific T cell engagers, ADCs, CAR expressing cells, cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some examples, the patient or subject is a validated animal model for disease, adoptive cell therapy, and/or for assessing toxic outcomes such as cytokine release syndrome (CRS).

In some embodiments, the subject has persistent or relapsed disease, e.g., following treatment with another immunotherapy and/or other therapy. In some embodiments, the administration effectively treats the subject despite the subject having become resistant to another therapy. In some embodiments, the subject has not relapsed but is determined to be at risk for relapse, such as at a high risk of relapse, and thus the compound or composition is administered prophylactically, e.g., to reduce the likelihood of or prevent relapse.

### Pharmaceutical Compositions

The compositions of the present invention may be administered in a number of ways depending upon whether local or systemic treatment is desired. In general, administration may be topical, parenteral, or enteral.

As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal.

Compositions of the present invention may be suited for topical, parenteral, or enteral administration. Preferably, formulated compositions comprising Abs, antigen-binding Ab fragments, ADCs, or CARs, polynucleotides or vectors encoding such, or cells expressing thereof are suitable for administration via parenteral administration for example, subcutaneous, intramuscular, intraperitoneal or intravenous injection.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release. Such formulation may be, for example, made of a biodegradable, biocompatible polymer, such as, but not limited to, ethylene vinyl acetate, poly(alkyl cyanoacrylates), poly(anhydrides), poly(amides), poly(ester), poly(ester amides), poly(phosphoesters), polyglycolic acid (PGA), collagen, polyorthoester, polylactic acid (PLA), poly(lactic-co-glycolidic acid) (PLAGA), or naturally occurring biodegradable polymers such as chitosan and hyaluronic acid-based polymers (Kamaly N. et al, Chem Rev. Author manuscript; available in PMC 2017 Jul. 13).

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams, microsponges, liposomes, nanoemulsions, aerosol foams, polymers, fullerenes, and powders. Conventional pharmaceutical carriers, aqueous, powder or oily bases, thickeners and the like may be necessary or desirable.

Compositions and formulations for parenteral, intrathecal, or intraventricular administration may include sterile aqueous solutions that may also contain buffers, diluents and other suitable additives such as, but not limited to, penetration enhancers, carder compounds and other pharmaceutically acceptable carriers or excipients.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated to provide appropriate in vivo distribution of the active ingredient. In many cases, concentrating the distribution of an anti-tumor drug in the tumor site is challenging, and it can be so even when a drug has a specificity to a molecule expressed by cancer cells. Various strategies have been developed to address the issue and any appropriate strategies may be applied for the current invention (for example, reviewed in Rosenblum D. et al., Nat Commun. 2018 Apr. 12; 9(1):1410. doi: 10.1038/s41467-018-03705-y). For delivering a drug to the brain, the drug needs to cross the blood-brain barrier (BBB). Any appropriate strategies to enable BBB crossing may be utilized to for the delivery of any of the NKp30 variants and conjugates containing (see for example, Dong X. et al., Theranostics. 2018; 8(6): 1481-1493, for exemplary strategies).

The compositions of the present invention may be formulated into any of many possible dosage forms such as, but not limited to, tablets, capsules, liquid syrups, soft gels, suppositories, aerosols, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Formulations comprising any of the NKp30 variants and conjugates containing of the present invention or cells expressing any of the NKp30 variants and conjugates containing of the present invention may include pharmaceutically acceptable excipient(s). Excipients included in the formulations will have different purposes depending, for example, on the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations comprising populations of the CAR-expressing cells of the present invention will typically have been prepared and cultured in the absence of any non-human components, such as animal serum (e.g., bovine serum albumin).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs. Such agents or drugs may be, but are not limited to, an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, or an imaging drug. Specific examples are, for instance, but are not limited to, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

### Kits

Also provided herein are kits comprising (a) one or more of NKp30 variants and conjugates containing (Ig fusion proteins, ADCs, CARs), polynucleotides encoding such, vectors encoding such, cells expressing such; and (b) for example an instruction for use in treating or diagnosing a disease or condition associated with NKp30 ligands such as B7H6. The kit may include a label indicating the intended use of the contents of the kit. The term “label” as used herein includes any written materials, marketing materials, or recorded materials supplied on, with, in, or appended to the kit.

### Method of Administration

The administration route used in the method of the present invention may be any appropriate route, which depends upon whether local or systemic treatment is desired.

In general, administration may be topical, parenteral, or enteral. Preferably, formulated compositions comprising Abs, antigen-binding Ab fragments, ADCs, or CARs, polynucleotides or vectors encoding such, cells expressing such may be administered parenterally, for example, via subcutaneous, intramuscular, intraperitoneal or intravenous injection.

In some embodiments, the composition of the present invention may be administered using any appropriate medical devices (for example, reviewed in Richter B. B., J. BioDrugs (2018) 32: 425).

### Dosing

For administration of any of the NKp30 variants and conjugates containing and compositions of the present invention, the dosage will vary and depend on, for example, the target disease, the severity of the disease, the route of administration, and pharmacokinetic factors. Dosing may be modified based on the response observed in the subject.

For administration of any of the NKp30 variants and conjugates containing, or compositions comprising such, appropriate dosage regimen may be determined using any appropriate methodology (for example, Bai S. et al., Clin Pharmacokinet. 2012 Feb. 1; 51(2):119-35. doi: 10.2165/11596370-000000000-00000).

In some embodiments, the dosage may be from about 1 ng/kg to about 1 g/kg (of the body weight of a subject) per day. In some embodiments, the dose may be from about 10 ng/kg/day to about 900 mg/kg/day, from about 20 ng/kg/day to about 800 mg/kg/day, from about 30 ng/kg/day to about 800 mg/kg/day, from about 40 ng/kg/day to about 700 mg/kg/day, from about 50 ng/kg/day to about 600 mg/kg/day, from about 60 ng/kg/day to about 500 mg/kg/day, from about 70 ng/kg/day to about 400 mg/kg/day, from about 80 ng/kg/day to about 300 mg/kg/day, from about 90 ng/kg/day to about 200 mg/kg/day, or from about 100 ng/kg/day to about 100 mg/kg/day. The treatment may be repeated or periodically given to a subject for days, months, or years, or until the desired effect is achieved. An exemplary dosing regimen include administering an initial dose of an NKp30 conjugate or ADC according to the invention of about 2 mg/kg, followed by a weekly maintenance dose of about 1 mg/kg.

Dosing frequency may be, for example, three times per day, twice per day, once per day, every other day, once per week, every other week, once per three weeks, once per four weeks, once per five weeks, once per six weeks, once per seven weeks, once per eight weeks, once per nine weeks, once per ten weeks, once per three months, once per four months, once per six months, once per year, or even less frequent.

The pharmaceutical composition in some embodiments contains cells expressing a CAR of the present invention in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells expressing an NKp30 variant or conjugate containing such as a CAR, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 103 to about 109 cells per kg body weight including all integer values of cell numbers within those ranges.

The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection into the disease site.

For purposes of the invention, the amount or dose of the inventive NKp30 variants and conjugates containing administered should be sufficient to effect a therapeutic or prophylactic response in the subject or animal over a reasonable time frame. For example, the dose of the inventive NKp30 variants and conjugates containing should be sufficient to bind to NKp30 ligand expressing cells such as B7H6, or detect, treat or prevent disease in a period of from about 2 hours or longer, e.g., about 12 to about 24 or more hours, from the time of administration. In certain embodiments, the time period could be even longer. The dose will be determined by the efficacy of the particular NKp30 variants and conjugates containing and the condition of the animal (e.g., human), as well as the body weight of the animal (e.g., human) to be treated.

In some embodiments, the NKp30 variants and conjugates containing or compositions of the present invention are administered as part of a combination treatment, such as simultaneously with or sequentially with, in any order, another therapeutic intervention, such as an antibody or engineered cell or receptor or agent, such as a cytotoxic or therapeutic agent. The cells or antibodies in some embodiments are co-administered with one or more additional therapeutic agents or in connection with another therapeutic intervention, either simultaneously or sequentially in any order. In some contexts, the NKp30 variants and conjugates containing or compositions are co-administered with another therapy sufficiently close in time such that the NKp30 variants and conjugates containing or compositions enhance the effect of one or more additional therapeutic agents, or vice versa. In some embodiments, the cells or antibodies are administered prior to the one or more additional therapeutic agents. In some embodiments, the NKp30 variants and conjugates containing are administered after the one or more additional therapeutic agents. Furthermore, the compositions of the present invention may be given to a subject along with one or more of other therapies, which may be surgery, or a radiotherapy.

In some embodiments, in CART therapy, a lymphodepleting chemotherapy is administered to the subject prior to, concurrently with, or after administration (e.g., infusion) of CAR cells. In an example, the lymphodepleting chemotherapy is administered to the subject prior to administration of the cells. For example, the lymphodepleting chemotherapy ends 1-4 days (e.g., 1, 2, 3, or 4 days) prior to CAR cell infusion. In embodiments, multiple doses of CAR cells are administered, e.g., as described herein. In embodiments, a lymphodepleting chemotherapy is administered to the subject prior to, concurrently with, or after administration (e.g., infusion) of a CAR-expressing cell described herein. Examples of lymphodepletion include, but may not be limited to, nonmyeloablative lymphodepleting chemotherapy, myeloablative lymphodepleting chemotherapy, total body irradiation, etc. Examples of lymphodepleting agents include, but are not limited to, anti-thymocyte globulin, anti-CD3 antibodies, anti-CD4 antibodies, anti-CD8 antibodies, anti-CD52 antibodies, anti-CD2 antibodies, TCRαβ blockers, anti-CD20 antibodies, anti-CD19 antibodies, Bortezomib, rituximab, anti-CD154 antibodies, rapamycin, CD3 immunotoxin, fludarabine, cyclophosphamide, busulfan, melphalan, Mabthera, Tacrolimus, alefacept, alemtuzumab, OKT3, OKT4, OKT8, OKT11, fingolimod, anti-CD40 antibodies, anti-BR3 antibodies, Campath-1H, anti-CD25 antibodies, calcineurin inhibitors, mycophenolate, and steroids, which may be used alone or in combination.

### Use as a Diagnostic Tool

The NKp30 variants and conjugates containing of the present invention, can be also useful as a diagnostic tool that may be used in vivo, ex vivo, or in vitro.

For example, an NKp30 variants and conjugates containing conjugated to an imaging agent may be administered to a subject or a patient to test if a diseased cell or tissue in the patient expresses NKp30 ligands such as B7H6. The diagnoses may be done using any imaging tools that can detect the imaging agent. Alternatively, a biological sample such as, but is not limited to, blood or biopsy sample, may be obtained, and an NKp30 variants and conjugates containing may be applied to the sample to test the expression of Nkp30 ligands such as B7H6.

These tests may determine whether the subject, or the cell or tissue of the subject, expresses NKp30 ligand such as B7H6 or not. In some embodiments, the test may determine whether the subject, or the cell or tissue of the subject, expresses sufficient amount of Nkp30 ligand such as B7H6 to be targeted by the NKp30 variant comprising therapeutic agent of the present invention. In some embodiments, the test may classify patients tumors based on different levels of NKp30 ligand, e.g., B7H6 expression, e.g., high-expressor, mid-expressor, or low-expressor.

In some embodiments, an appropriate therapeutic approach may be determined depending on the B7H6 expression. The expression may be determined using an anti-B7H6 antibody or an NKp30 variant of the present invention as described herein, or alternatively using any other appropriate method, such as, but not limited to, by measuring RNA expression levels or by quantifying B7H6 protein levels using an appropriate tool and/or technique.

### Variations

Included in the scope of the invention are functional modifications of the inventive NKp30 variant comprising agents described herein. A functional NKp30 variant modification can, for example, comprise the amino acid sequence of the parent NKp30 variant with at least one conservative amino acid substitution. Alternatively, or additionally, the modification can comprise the amino acid sequence of the parent with at least one non-conservative amino acid substitution. In this case, it is preferable for the non-conservative amino acid substitution to not interfere with or inhibit the biological activity of the functional NKp30 variant, i.e., its ability to bind B7H6 expressing cells and/or to elicit the expression of certain cytokines. The non-conservative amino acid substitution may enhance the biological activity of the functional variant, such that the biological activity of the functional variant is increased as compared to the parent.

Amino acid substitutions of the inventive NKp30 variants are preferably conservative amino acid substitutions. Conservative amino acid substitutions are known in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, lie, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a 3-branched side-chain substituted for another amino acid with a 3-branched side-chain (e.g., lie, Thr, and Val), an amino acid with an aromatic side-chain substituted for another amino acid with an aromatic side chain (e.g., His, Phe, Trp, and Tyr), etc.

Also, amino acids may be added or removed from the sequence based on vector design. The NKp30 variants can consist essentially of the specified amino acid sequence or sequences described herein, such that other components, e.g., other amino acids, do not materially change the biological activity of the functional variant.

Additionally such variants can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,ρ-diaminopropionic acid, homophenylalanine, and α-tert-butylglycine.

The experimental details of experiments further describing the invention are provided in the following examples. These examples are offered to illustrate, but not to limit, the claimed invention.

### EXAMPLES

### Abbreviations Used in Materials and Methods

B7H6—B7 homolog 6
BLI—Biolayer Interferometry
CAR—Chimeric Antigen Receptor
CRS—Cytokine Release Syndrome
FACS—Fluorescence Activated Cell Sorting
Fc—Fragment crystallizable
IL—Interleukin
MACS—Magnetic Activated Cell Sorting
MFI—Median Fluorescence Intensity
MHC—Major Histocompatibility Complex
MIP—Macrophage Inflammatory Protein
NK—Natural Killer
scFv—single chain Fragment variable
TNF-Tumor Necrosis Factor

### Materials and Methods

### Constructs, Protein Production and Purification

Soluble monomeric B7H6-His constructs were generated by cloning the extracellular B7H6 sequence into the pCMV expression plasmid, containing both a C-terminal Avi-tag and 6×His-tag. B7H6-Fc, TZ47-Fc, NKp30-Fc, CC3-Fc, and CC5-Fc were generated by fusing the extracellular domains to a mouse IgG2a Fc domain in pCMV expression vectors. Constructs were verified by Sanger sequencing. Fc-fusion proteins were expressed in expi293 HEK cells (Thermofisher) following the manufacturer's protocol. Soluble B7H6 with a 6×His-tag was purified via nickel-charged immobilized metal affinity chromatography (Thermofisher) according to the manufacturer's protocol; Fc-fusion recombinant proteins were purified from cell supernatants via Protein A resin-based affinity chromatography as previously described [25]. Eluates were then buffer exchanged into PBS using Amicon-30KD ultracentrifugation filters (Millipore).

### Directed Evolution of NKp30 by Yeast Surface Display

### Generation of NKp30 Library and Re-diversification

The extracellular domain of NKp30 was cloned as a C-terminal Aga2 fusion protein into the yeast expression vector pCHA [26]. The NKp30 extracellular domains in the pCHA vectors were mutagenized by salt-based error prone PCR [27]. Error-prone PCR was carried out with Taq polymerase (NEB) using the manufacturer's protocol. The generated PCR products were set up for a reamplification process via PCR to generate enough DNA for the yeast transformation process, again using Taq polymerase, with the same cycling conditions as for the initial error prone PCR. Yeast libraries were created by electroporation of the reamplified error-prone PCR products and digested pCHA vector. The error-prone PCR products were designed in a way such that the 5′ and 3′ ends of the PCR products shared homology with the digested vector. The digested vector and PCR products were transformed into EBY100 yeast following the electroporation transformation protocol, and cultured and induced as previously described [28]. Library diversity of the resulting g1.0 population was estimated by counting colonies from plated dilutions of the transformed yeast. Plasmids recovered from the g1.4 population using the ZymoPrep Yeast Plasmid Miniprep II Kit (Zymo Research) served as the template for another round of error-prone PCR as described above to introduce two to four random mutations for generation of population g2.0, which was transformed and enumerated as above.

### Magnetic-Activated Cell Sorting (MACS) of Yeast Libraries

Magnetic bead-based sorting of yeast libraries was conducted as described previously [29]. Briefly, B7H6-His was site-specifically biotinylated on the N-terminal avi-tag using the BirA biotin ligase and biotinylation kit, following instructions from the manufacturer's protocol (Avidity) for capture on Streptavidin beads. Streptavidin-coated magnetic beads (DynaBeads M-270) were washed five times with 0.1% PBSB (PBS+0.1% BSA) prior to incubating overnight with saturating amounts of biotinylated B7H6-His as determined through titration experiments. After saturation with biotinylated B7H6-His, the beads were washed 5× with PBSB to remove excess soluble protein prior to co-incubation with yeast libraries for 1.5 hrs at 4° C. Each MACS round included one negative selection using bare streptavidin magnetic beads and one positive selection using biotinylated B7H6-His-coated beads. To increase the likelihood of sampling the complete diversity available, a minimum of 10× the theoretical diversity of each population was employed in all culturing, induction, and selection steps. After each round of selection, beads were washed 1× with 1 mL PBSB for 30 min at 4° C. to separate bead-bound yeast from those that were trapped between beads (wash fraction). For g1.1 and following, aliquots of bead-bound and washed yeast were counted separately, but populations were regrown and combined in subsequent rounds of selection.

### Fluorescence-Activated Cell Sorting (FACS) of Yeast Libraries

For each round of FACS, at least 10× the estimated population diversity, or a minimum of 1 million, induced yeast was handled. Cells were spun down at 3000×g for 3 min and washed three times with 0.1% PBSB to remove cellular debris. Primary incubations comprised of an incubation with chicken anti-cMyc (Gallus) or mouse anti-HA (Invitrogen) to monitor full-length protein expression. Cells were also incubated with biotinylated B7H6-His at concentrations ranging from 1 μM to 1 nM depending upon the stringency and stage of selection, in a total volume of 100 μL. After primary incubation, cells were washed 3× with 200 μL 0.1% PBSB. Fluorescent staining with secondary reagents included a 30 min incubation with goat anti-chicken conjugated to AF488, Streptavidin-PE, or goat anti-mouse conjugated to AF647 (Life Technologies). After 30 minutes, the yeast were washed three times with 200 μL 0.1% PBSB before being resuspended in a final volume of 200 μL 0.1% PBSB. The labeled cells were either analyzed on the Miltenyi MACSQuant analyzer or sorted on a Sony iCyt Sorter. The top 0.5-1% of cells falling into a diagonal gate with enhanced B7H6 binding relative to their level of expression were selected using FlowJo.

### Structure Visualization and Manipulation

Receptor alignments were generated using Geneious. The sequence for human NKp30 was retrieved from the Protein Data Bank entry 3NOI. For NKp30 and B7H6 interaction, the protein data bank entry PDB:3PV6 was used. Structure visualizations were performed with UCSF Chimera, developed by the Resource for Biocomputing, Visualization, and Informatics at the University of California, San Francisco, with support from NIH P41-GM103311[30].

### Affinity and Avidity Assays

### BioLayer Interferometry for Affinity K_{D} Measurements

BioLayer Interferometry (BLI) measurements of affinity KD were obtained using the ForteBio Octet RED96 system as previously described [25]. Briefly, Protein A-coated biosensor tips were “activated” by incubation with 41 nM TZ47-Fc, NKp30-Fc, or CC3-Fc for 10 min to load tips. Binding to soluble B7H6-His were assessed by allowing TZ47-Fc, NKp30-Fc, or CC3-Fc coated tips to equilibrate in PBS+0.1% Tween-20 (PBST) for 5 min, followed by association steps in 200 μL of each antigen at concentrations ranging from 0 to 3000 nM for 10 min, and dissociation steps in 200 [symbol] L of PBST for 5 min. Data was analyzed using Octet® System Data Analysis software.

### Fluorescent Staining of B7H6-Expressing Cells

NKp30-Fc, CC3-Fc, CC5-Fc, or TZ47-Fc binding to B7H6-expressing cell lines was analyzed using a Miltenyi Biotech MACSQuant Flow Cytometer. Three different tumor types were stained with Ig-fusion proteins to determine avidity on varying levels of B7H6 expression. 96-well plates containing 2.5×10⁵ cells/well of K562 (ATCC® CCL-243™), A375 (ATCC® CRL-1619™), or Panc1 (ATCC® CRL-1469™) were washed twice with FBS Stain Buffer (BD #554656) before a primary incubation for 1 hour with varying concentrations of Fc-fusion proteins at concentrations ranging from 0 to 500 nM. Cells were then washed 3 times with Stain Buffer (PBS+2% FBS) before incubation with anti-Mouse Ig antibody conjugated to AF647 for 30 min at room temperature followed by a final wash. Live cell gates were drawn based on FSC vs SSC profiles and were used to calculate mean fluorescence intensity (MFI) values using FlowJo software. For staining of B7H6 expression, K563, A375, and Panc1 were stained with phycoerythrin-conjugated anti-human B7H6 (R&D #FAB7144P). PC3 (ATCC® CCL-1435™) and A549 (ATCC® CCL-185™) cell lines do not express B7H6 and were stained with anti-B7H6 as negative controls. 96-well plates containing 2.5×10⁵ cells/well of each tumor cell line were washed twice with FBS Stain Buffer before incubating with anti-B7H6 for 30 mins. Cells were then washed 3 times with Stain Buffer before flow cytometry analysis. Staining was overlayed with unstained samples of each cell type in FlowJo software.

### Construction and Transduction of CAR T Cells

The NKp30 CARs were constructed by PCR amplification of the natural receptor human NKp30, then subcloned using restriction cloning into pFB-Neo retroviral vectors containing the sequence for human CD28 cytoplasmic domain, human CD3ζ cytoplasmic domain, a T2A sequence, and a truncated form of mouse CD19. Vector control constructs were constructed similarly, with truncated mouse CD19 only in the pFB-Neo retroviral backbone. Constructs were verified by Sanger sequencing. NKp30 variants were generated as described above and subcloned into pFB-Neo retroviral vectors using restriction cloning.

### Retrovirus Production

To package retrovirus, 2.5×10⁶ HEK-293T cells were plated on a 10 cm plate in Dulbecco's modified Eagle's media (DMEM) 18 hrs before transfection. To produce ecotropic virus, cells were transfected using the calcium phosphate method with 10 μg of a B7H6-specific CAR plasmid and 10 μg of ψpcl-eco packaging plasmid. Media was replaced with fresh media ˜8 hrs post calcium phosphate treatment. Viral supernatants were harvested 48 hrs post transfection and filtered through a 0.45 μM filter before use for mouse CAR T cells or flash freezing and storing at −80° C. To make stable cell lines which produce amphotropic virus used for T cell transduction, ecotropic virus made from Hek-293T cells was used to infect PG13 packaging cells, followed by G418 selection (1 mg/mL) for 5 days. Amphotropic virus was collected, filtered, and used to generate human CAR T cells or stored as described above. All cell lines were cultured under 5% CO₂ at 37° C.

### Human CAR T Cells

Human PBMC from a deidentified and coded healthy donor cohort approved by the Dartmouth College Institutional Review Board were activated using anti-CD3 (40 ng/ml; OKT3 Biolegend) and cultured in complete RPMI media plus 100 U/ml of recombinant human IL-2 for 48 hrs. Complete RPMI media is Hyclone RPMI-1640 media supplemented with 10% heat inactivated FBS (Hyclone), 10 mM HEPES (Gibco), 0.1 mM non-essential amino acids (Gibco), 1 mM sodium pyruvate (Gibco), 100 U/mL penicillin (Hyclone), 100 μg/mL streptomycin, and 50 μM of 2-mercaptoethanol (Gibco). To prepare for retroviral transduction, non-tissue culture treated 24 well plates (Greiner Bio-One) were coated with 10 μg/mL retronectin (Takara) for 24 hrs at 4° C. and washed with PBS. To transduce activated T cells, 900 μL of viral supernatant containing 100 U/mL of IL-2 and 40 ng/ml OKT3 antibody were aliquoted into rectronectin coated wells prior to addition of 1×10⁶ T cells per well. Plates were spinoculated by centrifugation at 1500×g for 60 minutes at 30° C. Plates were then incubated at 37° C. for 24 hrs. The following day, 500 μL of media was removed from each well and replaced with new viral supernatant containing 100 U/mL of IL-2. Cells were then spinoculated again as above. After 24 hrs, T cells were resuspended in complete RPMI media containing 100 U/mL and cultured at 0.5-1×10⁶ cells/mL for 24 hrs. Two days post initial transduction, cells were analyzed by flow cytometry to detect CAR expression before experimental assays were performed.

### Mouse CAR T Cells

Mouse T cells from the spleens of C57Bl/6 mice were transduced 18-24 hrs after Concanavalin A (1 μg/ml; Sigma) stimulation and cultured in Complete RPMI media containing 25 U/ml of IL-2. Mouse T cells were transduced with the cytokine expressing backbone by resuspending activated T cells in ecotropic virus and spinning in 24 well plates with 8 million cells per well. Cells were spun at 1500 g for 90 mins at 37° C. Three days post-activation, cells were analyzed by flow cytometry to detect CAR expression before experimental set up.

### CAR T Cell Functional Assays

### Luciferase-Based Cytotoxicity

Luciferase-expressing human tumor cell lines A375, Panc1, and K562, and mouse cell lines B16-F10 and RMA with and without B7H6 expression were plated at 5×10³ cells per well in a black, tissue culture-treated, 96 well flat-bottom plate. CAR T cells were added at various T cell effector to target ratios (E:T) of 1:1 and 0.5:1. Cells were co-cultured at 37° C. for 24 hrs, followed by addition of 50 μL of luciferin (200 μg/mL) (Goldbio), and incubated at 37° C. for 30 mins before analyzing luminescence via a Centro LB960 Berthold Technologies luminometer. Supernatant was collected for multiplex cytokine analysis.

### Multiplex Cytokine Array

Levels of Interleukin (IL)-2, IL-4, IL-5, IL-6, IL-10, IL-13, IFN-γ, Granzyme B, Macrophage Inflammatory Proteins (MIP)-1a, and tumor necrosis factor (TNF)-α were determined using a MILLIPLEX MAP Human CD8+ T Cell Magnetic Bead Panel (Millipore) and measured using a Flexmap Luminex instrument and xponent software (version 4.2, Luminex) according to manufacturer's instructions. Based on previous optimization experiments, supernatant from cytotoxicity assays was analyzed at a 1:4 dilution.

### Data Analysis

A paired one-way ANOVA using Dunnett's multiple test correction was performed to analyze differences in target cell killing between NKp30 and other CARs. For analysis of cytokine expression, a paired one-way ANOVA with false discovery controlled using the Benjamini, Krieger, and Yekutieli two state step up method was performed. Statistical analysis was conducted in GraphPad Prism.

### Example 1: Isolation and Characterization of Yeast-Displayed NKp30 Variants

Directed evolution of NKp30 variants with enhanced binding affinity to B7H6 was conducted via yeast surface display, employing iterative diversification by error-prone PCR and magnetic and fluorescent cell sorting (FIG. 1A). Neither wildtype NKp30 (FIG. 1B) nor the initial library (not shown) showed detectible binding to B7H6-Fc by flow cytometry. Consequently, a bead-based Magnetic-assisted cell sorting (MACS) approach that leverages high avidity was used for initial selection, followed by fluorescence-activated cell sorting (FACS) with decreasing concentrations of either bivalent B7H6-Fc or monovalent B7H6-His to gradually increase selection stringency until clones with binding profiles similar to the TZ47 scFv were observed (FIG. 1B-C). Sequencing of selected clones identified a consensus mutation (S82P) at a position defined as a contact residue [31] that was conserved among all sequenced clones (FIG. 2A). Other mutations were observed both within and outside of the B7H6 binding interface (FIG. 2B), and one (T70A) eliminated an N-linked glycosylation motif.

### Example 2: Affinity, Avidity, and Specificity of Soluble NKp30-Fc Variants

Clone CC5, which contains only the consensus S82P mutation, along with CC3, which had two additional amino acid substitutions, were chosen for further characterization. To define binding affinities and kinetics with improved resolution, CC3, CC5, NKp30, and TZ47 sequences were expressed recombinantly as Fc fusion proteins. Affinity to monovalent B7H6 was determined by Biolayer Interferometry (BLI). Both CC3 and CC5 bound to B7H6 with a steady state affinity (KD) of 680 nM and 580 nM, respectively, representing a two-fold improvement compared to NKp30 (1.3 μM) (FIG. 3A). Interestingly, TZ47 bound B7H6 with a similar affinity (580 nM). However, this scFv exhibited drastically different B7H6 binding kinetics (kd and ka) compared to NKp30 and variants, with considerably slower on and off rates (FIG. 3B). The fast on and off rate profile of NKp30 was maintained in the variants despite the increase in overall affinity.

Next, we sought to measure the avidity and specificity of the newly generated NKp30 variants by testing their ability to bind to tumor cell lines expressing varying levels of B7H6. To characterize binding to native membrane-bound B7H6 antigen, three tumor cell lines which naturally express B7H6 at varying levels were stained with soluble NKp30-Fc, CC3-Fc, CC5-Fc, or TZ47-Fc. K562, a human leukemic cell line, expresses a high amount of B7H6 (B7H6^{high}), while A375, a human melanoma cell line, and Panc1, a human epithelial carcinoma cell line, express a lower amount of B7H6 (B7H6^{low}) [4] (FIG. 7). Despite showing a difference in monovalent affinity when measured with BLI, NKp30-Fc bound to K562 tumor cells with a similar avidity as the CC3-, CC5-, and TZ47-Fc fusion proteins (FIG. 4A). This result suggests that in the context of a bivalent Fc-fusion, the avidity from high antigen expression compensates for low affinity interactions. While it is unclear whether this phenomenon was related to differences in epitope [21], binding kinetics, or other factors, TZ47-Fc was unable to bind to A375 and Panc1 tumor cells, which express low levels of B7H6, whereas NKp30-, CC3-, and CC5-Fc were able to bind to both low B7H6 expressing tumor cell lines (FIG. 4A-B).

### Example 3: NKp30 Variant CAR T Cell Cytotoxicity

We next hypothesized that the difference in binding seen in the B7H6^{low} cell lines may confer a difference in CAR-T cell anti-tumor function. CAR constructs composed of NKp30, CC3, CC5, or TZ47-based extracellular domains and the intracellular domains of CD28 and CD3ζ were generated and expressed in primary human T cells from three donors (FIG. 8). These human CAR T cells were co-cultured with K562, A375, and Panc1 tumor cell lines expressing luciferase. CAR cytotoxicity was defined by the reduction of luminescence associated with target cell death after 24 hrs of co-culture (FIG. 4C). While CC3 and CC5 CAR T cells demonstrated similar killing potency as NKp30 against B716^{high} target cells, they demonstrated a modest increase in killing of B7H6^{low} tumor cells (FIG. 4D). This suggests that engineered natural receptors that bind with higher affinity to their tumor ligands can exhibit improvement in cytolytic activity against targets expressing low tumor antigen. Despite its lack of cell surface staining of low B7H6 cell lines as an scFv-Fc, TZ47 CAR T cells showed similar cytotoxicity as the engineered NKp30 variants, again suggesting the important role of avidity in CAR T cell activity. The killing activity of each CAR construct was confirmed to be antigen-dependent based on additional killing assays conducted using primary mouse T cells and targeting two mouse cell lines (RMA and B16) with and without engineering to express B7H6 (FIG. 9)

### Example 4: NKp30 Variants Drive Distinct Cytokine Profiles

In addition to direct cytolysis, CART cells produce an array of soluble effectors including cytokines and chemokines that are important for CAR T cell efficacy. To determine the effect of increased affinity on relative expression of soluble cytokines, we performed a multiplex cytokine assay on the supernatant collected from the killing experiments for each CAR construct and each of the B7H6-expressing tumor cell lines (FIG. 5). Cytokines were grouped into functional, stimulatory, regulatory, and inflammatory categories, essentially as defined by Xue et al., 2017 [32]. Among functional cytokines (FIG. 5A), although Granzyme B production was constitutively high in CAR T cells alone, IFNγ, TNFα, and to a lesser extent, MIP-1α, were all induced by co-culture with tumor cells. NKp30 variants generally exhibited higher IFNγ production than either NKp30 or TZ47. For B7H6^{low} Panc1 and A375 target cells, the differences between NKp30 and its variants were more apparent, with higher MIP-1α, TNFα and IFNγ secreted by the NKp30 variants compared to wildtype. However, for B7H6^{high} K562 target cells, the variants showed similar or somewhat decreased production of functional cytokines compared to wildtype. This trend was also observed with the stimulatory cytokines, GM-CSF and IL-2 (FIG. 5B), and to a lesser degree with the regulatory cytokines IL-5 and IL-13 (FIG. 5C). In contrast, IL-10 and IL-4 remained largely unchanged with or without tumor cell co-culture. This pattern was not observed with the single inflammatory cytokine tested, IL-6 (FIG. 5D). IL-6 was constitutively expressed by K562 and A375 target cells and varied considerably across different target cell co-cultures, with CAR T cells cultured with Panc1 cells showing limited to no expression, A375 cells inducing high amounts of IL-6 across all CAR constructs, and K562 showing an intermediate phenotype and greater expression induced by NKp30 than either of its engineered variants or TZ47. Collectively, these cytokine expression profiles highlight the role that target cell diversity can play in cytokine expression beyond the impact of CAR-dependent factors. To this end, target cells expressing low levels of B7H6 drove increased expression of cytokines considered to be desirable from T cells expressing the engineered variants relative to NKp30 (FIG. 6). With the exception of the Panc1 cells that had low levels of basal IL-6 expression, this elevation was not observed for the cytokine release syndrome-associated IL-6, suggesting that desirable and undesirable T cell profiles may be separable and that even subtle changes in antigen recognition have the potential to influence soluble mediators of CAR T cell activity.

### Example 5: In Vivo Experiments Using Car Cells which Express NKp30 Variants

In vivo experiments were conducted in order to confirm the advantageous properties of NK p30 variants as disclosed herein. The following Materials and Methods were used.

### Materials & Methods

### DNA Constructs

In these experiments a pFB backbone was used for the NKp30 CAR constructs which included the CAR sequence, a T2A, followed by a truncated mouse CD19 sequence in which all signaling components were removed to act as a marker for transduction.

### Generation of CAR T Cells

Splenocytes from B6 mice were stimulated for 18-24 hrs with Concanavalin A (1 μg mL-1; Sigma) and cultured in Complete RPMI media plus 25 U mL-1 of IL-2. T cells were transduced with retrovirally encoded NKp30 supernatant by resuspending activated T cells in viral supernatant and polybrene (1 ug mL-1; Sigma) at 8 million cells per well in 24 well plates followed by spinoculation at 1500×g for 90 mins at 37° C.

### In Vivo Tumor Experiments

C57BL/6 mice were injected with 1×10⁵ RMA cells expressing B7H6 in 50 μl of HBSS via intravenous (i.v.) tail vein injections. Tumors were established in mice for 7 days and then treated with 7×10⁶ NKp30 CAR T cells via intravenous (i.v.) tail vein injections. Tumor burden was detected by IVIS luminescence imaging on the indicated days.

### Results

The experimental results in FIG. 10 show Nkp30 in vivo transduction of CD19 and NKp30 in mice treated with WT NKp30, variant (CC3) NKp30 and JC32 control. (JC32 is Tz47 28 CAR in PFB Neo which provides a direct comparison to the other NKp30 CAR constructs).

The experimental results in FIG. 11 compare tumor growth of RMA B7H6 tumors in NKp30 WT, NKp30 CC3 variant and TZ47 CAR treated and control (no treatment) mice. The results suggest (in view of results in FIG. 10) that the improved reduction of tumor size which is seen in the mice transduced with the CAR comprising the variant (CC3) NKp30 construct be due to transduction differences.

The experimental results in FIG. 12 are IVIF tumor images at days 6, 8, 10, 12, 14 and 17 of NKp30 WT, NKp30 CC3, and T247 CAR treated, and control (no treatment) RMA B7H6 tumor bearing mice.

The experimental results in FIG. 13 are survival data showing that RMA B7H6 tumor mice treated with the CC3 NKp30 mutant have significantly increased survival time compared to the survival time of NKp30 WT treated RMA B7H6 tumor bearing mice.

These in vivo results further indicate that the inventive NKp30 variants when incorporated into CARS or other conjugates should provide for enhanced anti-tumor efficacy when used as therapeutics compared to CARS comprising wild-type NKp30 sequences and may facilitate treatment of tumors wherein the expression of B7H6 ligands is moderate or low.

### CONCLUSIONS

We sought to determine the effect of directed evolution of a natural receptor for improved antigen recognition on in vitro CAR T cell function. A set of single- and double-point mutations to the stress receptor NKp30 that permitted a high level of antigen binding in the context of yeast display resulted in a two-fold increase in binding affinity to recombinant tumor-associated antigen B7H6. This affinity increase was associated with little difference in the ability to bind B7H6 expressed on tumor cells when tested in the context of an Fc fusion. In contrast, some improvement in killing of tumor cells with low but not high antigen expression was observed when tested in the context of a CAR T cell. These observations suggest that high avidity can substitute for high affinity in the context of high antigen expression, but that affinity may play a role in the efficiency of killing target cells with low levels of antigen expression. To this end, it has been shown previously that low affinity interactions can allow for discrimination between tumor cells with high antigen expression and normal tissues with lower expression, thus decreasing on-target, off-tumor toxicity [33]. In instances of high CAR or high tumor antigen expression, avid interactions can overcome low affinity, effectively neutralizing small receptor:ligand affinity differences [34]. Indeed, it has long been theorized that in the case of TCR:MHC− peptide binding, there is an optimal threshold of affinity, and exceeding this threshold does not benefit T cell activation or signaling [35]. However, it has been shown that in some instances CARs with OM affinity can be superior to those with nM affinity [36, 37]. Our data suggests that the native NKp30 affinity to B7H6 is sufficient for cytotoxic functionality in T cells, but can still be modestly improved by modest increases in affinity.

Beyond killing, CAR signal strength has been shown to be a key determinant of T cell fate [38]. To this end, we measured a panel of cytokines in order to determine if tumor epitope and affinity differences could affect functional outputs in T cells. We found notable differences in cytokine output between NKp30 and its variants. While no single cytokine signature is yet predictive of in vivo outcomes, evidence for some relationships has accrued. For example, IL-2 is well characterized as a T cell growth factor and CARs expressing higher levels of IL-2 are likely to survive in the tumor microenvironment [39, 40]. We found higher affinity of NKp30 variants to B7H6 led to higher production of IL-2. We also observed increases in induction of TNFα and IFNγ, both of which have been shown to correlate with delayed tumor relapse in vivo [41], in CC3 and CC5 compared to wildtype in B7H6^{low} tumor cell lines. In contrast, expression of IL-6, a cytokine found to be predictive of severe cytokine release syndrome (CRS) [42, 43], was not robustly increased for engineered variants as compared to NKp30. However, IL-6 has also been found to be produced by myeloid cells in CRS; in vivo experiments in models capable of observing this toxicity [44] are needed to investigate whether these constructs impact this aspect of IL-6-mediated CRS. These results suggest that while a modest increase in affinity had a small effect on binding and killing functionality, it resulted in more marked differences in cytokine output. Most notably, these differences in cytokine output were most distinct with the B7H6^{low} cell lines, underscoring that increased affinity of NKp30 may be more advantageous against tumors with low B7H6 expression.

Notable distinctions between NKp30-based recognition of B7H6 and that of the TZ47 scFv were observed. Though this antibody fragment has a similar equilibrium binding affinity as the engineered NKp30 variants, it recognizes a different epitope. Whereas NKp30 binds to a distal site on B7H6, TZ47 recognizes a membrane proximal site [21], which has been reported to enhance the antitumor activity of CARs [45]. An Fc fusion of TZ47 failed to stain two tumor cell lines with low B7H6 expression; NKp30-based Fc fusions reliably stained these targets. This difference raises the possibility that the NKp30 epitope on B7H6 may be more favorable or accessible to binding in some contexts, and that bivalent binding may be achieved by recognition of some but not other epitopes of B7H6 when expressed at low levels. For a number of tumor-associated antigens, recognition of cells with low expression can lead to on-target, off-tumor toxicity. While it is possible that effective stimulation of CAR T cells by low levels of B7H6 could increase the risk of toxicity, this concern is tempered by its biological role as a stress marker and the absence of evidence of expression on healthy cells. Despite the lack of binding of bivalent TZ47-Fc, a TZ47-based CARs showed excellent tumor killing, in some cases exhibiting the greatest cytotoxicity. This observation, too, points to the complex interplay of affinity, avidity, and epitope specificity in tumor recognition and clearance,

As compared to scFvs, which can be isolated in principle against any tumor antigen epitope, and are often affinity matured in vitro [46], natural receptors bind to established epitopes which have been evolutionarily selected for functional ligation, but are typically treated as modular CAR extracellular domain units and not modified in sequence. Here, by evolving a natural receptor for use in CAR platforms, some limitations, such as the instability and tonic signaling that can be associated with scFvs may be avoided, while advantages of natural receptor recognition could be maintained. One of these advantages may be the fast on- and off-rates we observed for NKp30 and its variants, which are in stark contrast to the approximately 100-fold slower on and off-rates observed for TZ47, a characteristic most CAR scFvs exhibit [47]. Despite similar equilibrium affinity and somewhat poorer in vitro cytotoxicity, engineered NKp30 variants showed elevated expression of diverse functional and stimulatory cytokines as compared to TZ47, especially when B7H6 expression on target cells was low. These results suggest that natural receptor- and scFv-based CARs that bind the same tumor antigen with similar affinity can produce different signaling outputs, allowing for the potential to fine tune this parameter during CAR domain development. Whether an optimal activity has been reached, or whether continued increases in affinity would result in further improvement in CAR T cell function is not known, but is of interest given the complex relationships known to exist between affinity and activity. Additionally, further studies are necessary to investigate the relevance of these phenotypes to tumor clearance in vivo.

Like other natural receptors, NKp30 has been reported to interact with multiple ligands. Beyond B7H6, NKp30 is known to interact with several other stress-induced ligands. While we cannot exclude that these ligands play a role in the phenotypes that we have observed, at least one, BAT3 (BAG6) is reported to interact with a different site of NKp30, and is therefore not expected to show the same enhancement of binding. Unlike B7H6, BAT3 is not a well characterized tumor antigen, and it's ligation has been reported to be alternatively immune stimulating or immune inhibitory [48, 49]. Whether or not the potentially promiscuous binding of B7H6 to other NK cell ligands is beneficial or detrimental is unknown. Future investigation into this area could include mutational studies to eliminate interactions with receptors to which binding is undesirable.

In sum, we observed the impacts of modifying the affinity of tumor recognition can extend beyond killing to drive differential cytokine expression profiles suggesting that further engineering can be attempted to improve CAR therapy to more broadly influence cytokine, chemokine, or regulatory interactions. These observations suggest the value of thorough exploration of cytokine profiles in other contexts. Reducing or enhancing interactions to fine tune both recognition of target cells and manipulate signaling has the potential to significantly affect the outcome of cell-based therapies. As molecular engineering strategies are deployed to improve upon evolved interactions, we anticipate that additive advantages can be combined to propel future therapeutics forward.

### REFERENCES CITED IN THIS APPLICATION

1. Ahmad Z A, Yeap S K, Ali A M, et al (2012) ScFv antibody: Principles and clinical application. Clin. Dev. Immunol. 2012
2. Song D G, Ye Q, Santoro S, et al (2013) Chimeric NKG2D CAR-expressing T cell-mediated attack of human ovarian cancer is enhanced by histone deacetylase inhibition. Hum Gene Ther 24:295-305. https://doi.org/10.1089/hum.2012.143
3. Nikiforow S, Werner L, Murad J, et al (2016) Safety Data from a First-in-Human Phase 1 Trial of NKG2D Chimeric Antigen Receptor-T Cells in AML/MDS and Multiple Myeloma. Blood 128:4052-4052. https://doi.org/10.1182/blood.v128.22.4052.4052
4. Zhang T, Wu M-R, Sentman C L (2012) An NKp30-Based Chimeric Antigen Receptor Promotes T Cell Effector Functions and Antitumor Efficacy In vivo. J Immunol 189:2290-2299. https://doi.org/10.4049/jimmunol.1103495
5. Barber A, Rynda A, Sentman C L (2009) Chimeric NKG2D Expressing T Cells Eliminate Immunosuppression and Activate Immunity within the Ovarian Tumor Microenvironment. J Immunol 183:6939-6947. https://doi.org/10.4049/jimmunol.0902000
6. Shaffer D R, Savoldo B, Yi Z, et al (2011) T cells redirected against CD70 for the immunotherapy of CD70-positive malignancies. Blood 117:4304-4314. https://doi.org/10.1182/blood-2010-04-278218
7. Zhang T, Lemoi B A, Sentman C L (2005) Chimeric NK-receptor-bearing T cells mediate antitumor immunotherapy. Blood 106:1544-1551. https://doi.org/10.1182/blood-2004-11-4365
8. M R W, T Z, A A, C L S (2015) DNAM-1-based chimeric antigen receptors enhance T cell effector function and exhibit in vivo efficacy against melanoma. Cancer Immunol Immunother 64: https://doi.org/10.1007/S00262-014-1648-2
9. Willuda J, Honegger A, Waibel R, et al (1999) High Thermal Stability Is Essential for Tumor Targeting of Antibody Fragments: Engineering of a Humanized Anti-epithelial Glycoprotein-2 (Epithelial Cell Adhesion Molecule) Single-Chain Fv Fragment Cancer Res. 59, 5758-5767, Nov. 15, 1999]
10. Chang Z N L, Chen Y Y (2017) CARs: Synthetic Immunoreceptors for Cancer Therapy and Beyond. Trends Mol. Med. 23:430-450
11. Long A H, Haso W M, Shern J F, et al (2015) 4-1BB costimulation ameliorates T cell exhaustion induced by tonic signaling of chimeric antigen receptors. Nat Med 21:581-590. https://doi.org/10.1038/nm.3838
12. Ajina A, Maher J (2018) Strategies to address chimeric antigen receptor tonic signaling. Mol. Cancer Ther. 17:1795-1815
13. Zajc C U, Salzer B, Taft J M, et al (2020) Driving CARs with alternative navigation tools—the potential of engineered binding scaffolds. FEBS J.
14. Landoni E, Fuca G, Wang J, et al (2021) Modifications to the Framework Regions Eliminate Chimeric Antigen Receptor Tonic Signaling. Cancer Immunol Res. https://doi.org/10.1158/2326-6066.cir-20-0451
15. Salzer B, Schueller C M, Zajc C U, et al (2020) Engineering AvidCARs for combinatorial antigen recognition and reversible control of CAR function. Nat Commun 11: https://doi.org/10.1038/s41467-020-17970-3
16. Brandt C S, Baratin M, Yi E C, et al (2009) The B7 family member B7-H6 is a tumor cell ligand for the activating natural killer cell receptor NKp30 in humans. J Exp Med 206:1495-1503. https://doi.org/10.1084/jem.20090681
17. Kaifu T, Escaliere B, Gastinel L N, et al (2011) B7-H6/NKp30 interaction: A mechanism of alerting NK cells against tumors. Cell. Mol. Life Sci. 68:3531-3539
18. Salimi M, Xue L, Jolin H, et al (2016) Group 2 Innate Lymphoid Cells Express Functional NKp30 Receptor Inducing Type 2 Cytokine Production. J Immunol 196:45-54. https://doi.org/10.4049/jimmunol.1501102
19. Rusakiewicz S, Nocturne G, Lazure T, et al (2013) Sjögren's syndrome: NCR3/NKp30 contributes to pathogenesis in primary Sjögren's syndrome. Sci Transl Med 5:195ra96. https://doi.org/10.1126/scitranslmed.3005727
20. Fiegler N, Textor S, Arnold A, et al (2013) Downregulation of the activating NKp30 ligand B7-H6 by HDAC inhibitors impairs tumor cell recognition by NK cells. Blood 122:684-693. https://doi.org/10.1182/blood-2013-02-482513
21. Hua C K, Gacerez A T, Sentman C L, Ackerman M E (2017) Development of unique cytotoxic chimeric antigen receptors based on human scFv targeting B7H6. Protein Eng Des Sel 30:713-721. https://doi.org/10.1093/protein/gzx051
22. Wu M R, Zhang T, DeMars L R, Sentman C L (2015) B7H6-specific chimeric antigen receptors lead to tumor elimination and host antitumor immunity. Gene Ther 22:675-684. https://doi.org/10.1038/gt.2015.29
23. Hudecek M, Lupo-Stanghellini M T, Kosasih P L, et al (2013) Receptor affinity and extracellular domain modifications affect tumor recognition by ROR1-specific chimeric antigen receptor T cells. Clin Cancer Res 19:3153-3164. https://doi.org/10.1158/1078-0432.CCR-13-0330
24. Chmielewski M, Hombach A, Heuser C, et al (2004) T Cell Activation by Antibody-Like Immunoreceptors: Increase in Affinity of the Single-Chain Fragment Domain above Threshold Does Not Increase T Cell Activation against Antigen-Positive Target Cells but Decreases Selectivity. J Immunol 173:7647-7653. https://doi.org/10.4049/jimmunol.173.12.7647
25. Choi Y, Hua C, Sentman C L, et al (2015) Antibody humanization by structure-based computational protein design. MAbs 7:1045-1057. https://doi.org/10.1080/19420862.2015.1076600
26. Angelini A, Chen T F, De Picciotto S, et al (2015) Protein engineering and selection using yeast surface display. Methods Mol Biol 1319:3-36. https://doi.org/10.1007/978-1-4939-2748-7_1
27. Fromant M, Blanquet S, Plateau P (1995) Direct Random Mutagenesis of Gene-Sized DNA Fragments Using Polymerase Chain Reaction. Anal Biochem 224:347-353. https://doi.org/10.1006/abio.1995.1050
28. Chao G, Lau W L, Hackel B J, et al (2006) Isolating and engineering human antibodies using yeast surface display. Nat Protoc 1:755-768. https://doi.org/10.1038/nprot.2006.94
29. Ackerman M, Levary D, Tobon G, et al (2009) Highly avid magnetic bead capture: An efficient selection method for de novo protein engineering utilizing yeast surface display. Biotechnol Prog 25:774-783. https://doi.org/10.1002/btpr.174
30. Pettersen E F, Goddard T D, Huang C C, et al (2004) UCSF Chimera—A visualization system for exploratory research and analysis. J Comput Chem 25:1605-1612. https://doi.org/10.1002/jcc.20084
31. Li Y, Wang Q, Mariuzza R A (2011) Structure of the human activating natural cytotoxicity receptor NKp30 bound to its tumor cell ligand B7-H6. J Exp Med 208:703-714. https://doi.org/10.1084/jem.20102548
32. Xue Q, Bettini E, Paczkowski P, et al (2017) Single-cell multiplexed cytokine profiling of CD19 CAR-T cells reveals a diverse landscape of polyfunctional antigen-specific response. J Immunother Cancer 5: https://doi.org/10.1186/s40425-017-0293-7
33. Liu X, Jiang S, Fang C, et al (2015) Affinity-tuned ErbB2 or EGFR chimeric antigen receptor T cells exhibit an increased therapeutic index against tumors in mice. Cancer Res 75:3596-3607. https://doi.org/10.1158/0008-5472.CAN-15-0159
34. Walker A J, Majzner R G, Zhang L, et al (2017) Tumor Antigen and Receptor Densities Regulate Efficacy of a Chimeric Antigen Receptor Targeting Anaplastic Lymphoma Kinase. Mol Ther 25:2189-2201. https://doi.org/10.1016/j.ymthe.2017.06.008
35. Stone J D, Chervin A S, Kranz D M (2009) T-cell receptor binding affinities and kinetics: impact on T-cell activity and specificity. Immunology 126:165-176. https://doi.org/10.1111/.1365-2567.2008.03015.x
36. Ghorashian S, Kramer A M, Onuoha S, et al (2019) Enhanced CAR T cell expansion and prolonged persistence in pediatric patients with ALL treated with a low-affinity CD19 CAR. Nat Med 25:1408-1414. https://doi.org/10.1038/s41591-019-0549-5
37. Park S, Shevlin E, Vedvyas Y, et al (2017) Micromolar affinity CAR T cells to ICAM-1 achieves rapid tumor elimination while avoiding systemic toxicity. Sci Rep 7: https://doi.org/10.1038/s41598-017-14749-3
38. Salter A I, Ivey R G, Kennedy J J, et al (2018) Phosphoproteomic analysis of chimeric antigen receptor signaling reveals kinetic and quantitative differences that affect cell function. Sci Signal 11: https://doi.org/10.1126/scisignal.aat6753
39. Qasim W, Zhan H, Samarasinghe S, et al (2017) Molecular remission of infant B-ALL after infusion of universal TALEN gene-edited CAR T cells. Sci Transl Med 9: https://doi.org/10.1126/scitranslmed.aaj2013
40. Gillis S, Watson J (1980) Biochemical and biological characterization of lymphocyte regulatory molecules: V. Identification of an interleukin 2-producin human leukemia T cell line. J Exp Med 152:1709-1719. https://doi.org/10.1084/jem.152.6.1709
41. Ma C, Cheung A F, Chodon T, et al (2013) Multifunctional T-cell analyses to study response and progression in adoptive cell transfer immunotherapy. Cancer Discov 3:418-429. https://doi.org/10.1158/2159-8290.CD-12-0383
42. Pabst T, Joncourt R, Shumilov E, et al (2020) Analysis of IL-6 serum levels and CAR T cell-specific digital PCR in the context of cytokine release syndrome. Exp Hematol 88:7-14.e3. https://doi.org/10.1016/j.exphem.2020.07.003
43. Teachey D T, Lacey S F, Shaw P A, et al (2016) Identification of predictive biomarkers for cytokine release syndrome after chimeric antigen receptor T-cell therapy for acute lymphoblastic leukemia. Cancer Discov 6:664-679. https://doi.org/10.1158/2159-8290.CD-16-0040
44. van der Stegen S J C, Davies D M, Wilkie S, et al (2013) Preclinical In vivo Modeling of Cytokine Release Syndrome Induced by ErbB-Retargeted Human T Cells: Identifying a Window of Therapeutic Opportunity? J Immunol 191:4589-4598. https://doi.org/10.4049/jimmunol.1301523
45. Zhang Z, Jiang D, Yang H, et al (2019) Modified CAR T cells targeting membrane-proximal epitope of mesothelin enhances the antitumor function against large solid tumor. Cell Death Dis 10:1-12. https://doi.org/10.1038/s41419-019-1711-1
46. Bradbury A (2003) scFvs and beyond. In: Drug Discovery Today. Drug Discov Today, pp 737-739
47. Sasmal D K, Feng W, Roy S, et al (2020) TCR-pMHC bond conformation controls TCR ligand discrimination. Cell Mol Immunol 17:203-217. https://doi.org/10.1038/s41423-019-0273-6
48. Binici J, Hartmann J, Herrmann J, et al (2013) A soluble fragment of the tumor antigen BCL2-associated athanogene 6 (BAG-6) is essential and sufficient for inhibition of NKp30 receptor-dependent cytotoxicity of natural killer cells. J Biol Chem 288:34295-34303. https://doi.org/10.1074/jbc.M113.483602
49. Binici J, Koch J (2014) BAG-6, a jack of all trades in health and disease. Cell. Mol. Life Sci. 71:1829-1837

Although various embodiments and examples of the present invention are described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims.

All references cited herein, including patent documents and non-patent documents, are hereby incorporated by reference in their entirety. Nothing herein is to be construed as an admission that the present invention is not entitled to antedate such disclosure by virtue of prior invention.

Having described the invention in detail, the invention is further characterized in the claims which follow.

We claim the following:

1. A human NKp30 variant polypeptide that (i) binds to B7H6 with higher affinity compared to the native NKp30 receptor polypeptide and which (ii) optionally retains its fast association and/or (iii) dissociation profile and/or (iv) elicits a different cytokine profile than native NKp30 or TZ47 and/or (v) exhibits improved in vitro or in vivo tumor cell killing relative to native NKp30 and/or (vi) binds to tumor cells expressing low levels of B7H6 better than native NKp30 or TZ47.

2. The human NKp30 variant of claim 1, or a fusion protein, conjugate, chimeric antigen receptor, binding agent drug conjugate (ADC), bispecific T cell engager, or multispecific binding polypeptide comprising said human NKp30 variant, wherein:
(a) the extracellular domain of NKp30 is modified to include at least one of the following modifications in the extracellular domain: (i) G at position 26, (ii) P at position 52; (iii) W or S at position 67; (iv) A at position 70; (v) G at position 74; (vi) P at position 82; (vii) D at position 91; or (viii) G at position 104;
(b) the extracellular domain of NKp30 is modified to include at least one of the following modifications: (i) A at position 70; (ii) G at position 104 or (iii) P at position 82;
(c) the extracellular domain comprises the same or additional mutations as CC3 or CC5 as shown in FIG. 2;
(d) it comprises a detectable label;
(e) it comprises an effector moiety;
(f) it comprises an effector moiety selected from an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a radioisotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, or an imaging drug;
(g) it comprises one or more protein domains which facilitate or enhance stable protein expression, or enhanced or altered signal transduction in immune cells, optionally T or NK cells;
(h) it comprises one or more protein domains which facilitate or enhance stable protein expression, or enhanced or altered signal transduction in immune cells, optionally T or NK cells which are selected from Fc domains or human CD3Z, CD28, Dap10, CD27, and CD8 domains;
(i) it comprises a drug or effector moiety which is selected from an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a radioisotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, or an imaging drug;
(j) it comprises an Fc or albumin protein, further optionally a human IgG1, IgG2, IgG3 or IgG4 Fc fusion protein which Fc is optionally mutated to impair or enhance at least one Fc-associated effector function;
(k) it comprises a CAR which comprises at least one signaling moiety;
(l) it comprises at least one other binding moiety;
(m) it comprises at least one other binding moiety which comprises an antibody or antibody fragment which specifically binds to a target antigen or epitope;
(n) it comprises at least one other binding moiety which comprises an antibody or antibody fragment which specifically binds to a an antigen expressed on an immune cell, further optionally a B, NK cell, Treg, T effector cell, CTL, dendritic cell, neutrophil, macrophage, monocyte, myeloid cell, eosinophil, or a precursor of any of the foregoing, further optionally wherein the antigen is CD3, PD-1, PD-L1, PD-L2, CTLA-4, CD28 or B7H6;
(o) it comprises at least one other binding moiety which comprises an antibody or antibody fragment which specifically binds to a an antigen expressed on one or more of a B, NK cell, Treg, T effector cell, CTL, dendritic cell, neutrophil, macrophage, monocyte, myeloid cell, eosinophil, or a precursor of any of the foregoing;
(p) it comprises at least one other binding moiety which comprises an antibody or antibody fragment which specifically binds to CD3, PD-1, PD-L1, PD-L2, CTLA-4, CD28 or B7H6;
(p) it comprises signaling domains;
(q) it comprises CD28 and CD3ζ intracellular domains;
(r) it comprises a CAR which comprises: (i) a NKp30 variant according to any of the foregoing, (ii) a transmembrane (TM) domain, and optionally (iii) an intracellular signaling (ICS) domain and further optionally, a (iv) a hinge that joins said NKp30 variant and said TM domain, and further optionally (v) one or more costimulatory (CS) domains;
(s) it comprises a CAR according to (r) which comprises an ICS domain, optionally a cytoplasmic signaling sequence, or a functional fragment thereof;
(t) it comprises a CAR according to (r) or (s) which comprises one or more ICS domains selected from CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, or DAP12 and/or the ICS domain comprises a CD3z cytoplasmic signaling sequence, or a functional fragment thereof or a CD28 hinge;
(u) it comprises a CAR according to (r), (s) or (t) which comprises one or more CS domains derived from a cytoplasmic signaling sequence, or functional fragment thereof;
(v) it comprises a CAR according to (r), (s) or (t) which comprises one or more CS domains derived from a cytoplasmic signaling sequence, or functional fragment thereof selected from CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAM1, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand; and/or comprises a CS domain derived from a cytoplasmic signaling sequence of CD28, 4-1BB, or DAP10, or functional fragment thereof;
(w) it comprises a cytotoxic drug which is directly or indirectly conjugated to the NKp30 variant; or
(x) any combination of (a) to (w).

3-19. (canceled)

20. An isolated polynucleotides or and/or a combination of polynucleotides vector which separately or in combination encode a NKP30 variant or CAR, fusion protein, conjugate, bispecific T cell engager, multispecific binding polypeptide, or ADC comprising an NKp30 variant according to claim 2.

21. An isolated polynucleotide or the combination of isolated polynucleotides according to claim 20, which
(a) further encodes an antibody (Ab) or antigen-binding Ab fragment, which optionally may comprise a VH and a VL, further optionally a monoclonal Ab, a monospecific Ab, a bispecific Ab, a multispecific Ab, a humanized Ab, a tetrameric Ab, a tetravalent Ab, a single chain Ab, a domain-specific Ab, a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a diabody, or a minibody, which antibody optionally binds to B7H6 or another antigen, further optionally another antigen expressed on an immune cell-;
(b) further encodes an Fc region optionally derived from the Fc region of a human IgM, a human IgD, a human IgG, a human IgE, or a human IgA, optionally of a human IgG1, a human IgG2, a human IgG3, or a human IgG4; optionally wherein the human or human-like Fc region binds to an Fc receptor (FcR), optionally an Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn);
(c) further encodes an (a) an AB domain that binds to a target antigen, e.g., tumor antigen or immune cell antigen; (b) a transmembrane (TM) domain; (c) an intracellular signaling (ICS) domain; (d) optionally a hinge that joins said AB domain and said TM domain; and (e) optionally one or more costimulatory (CS) domains;
(d) encodes a CAR comprising said NKp30 variant which comprises at least one ICS domain derived from a cytoplasmic signaling sequence, or a functional fragment thereof, optionally that of CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12;
(e) encodes a CAR which comprises at least one CS domain derived from a cytoplasmic signaling sequence, or functional fragment thereof, optionally that of CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMF1, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAM1, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand;
(f) encodes a CAR which comprises at least one CS domain derived from a cytoplasmic signaling sequence of CD28, 4-1BB, or DAP10, or functional fragment thereof; or
(g) any combination of (a) to (f); or
a vector or vectors or vectors comprising said isolated polynucleotide or combination of isolated polynucleotides, optionally a DNA, an RNA, a plasmid, a cosmid, a viral vector, a lentiviral vector, an adenoviral vector, or a retroviral vector.

22-27. (canceled)

28. A cell or cells, optionally immune cell(s), which express an NKp30 variant or a fusion protein, conjugate, bispecific T cell engager or CAR comprising a NKp30 variant according to claim 2.

29. The cell or cells of claim 28, which:
(i) comprises a T, B, NK cell, Treg, T effector cell, CTL, dendritic cell, neutrophil, macrophage, monocyte, myeloid cell, eosinophil, or a precursor of any of the foregoing;
(ii) comprises a non-mammalian cell, optionally a plant cell, a bacterial cell, a fungal cell, a yeast cell, a protozoa cell, or an insect cell;
(iii) comprises a mammalian cell, optionally a human cell, a rat cell, or a mouse cell;
(iv) comprises a stem cell;
(v) comprises a primary cell, optionally a human primary cell or derived therefrom;
(vi) comprises a hybridoma cell line;
(vii) comprises an immune cell;
(viii) is MHC+ or MHC−;
(ix) comprises a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH3 cell, a TH17 cell, a TH9 cell, a TH22 cell, a follicular helper T cells, and a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, or a dendritic cell;
(x) comprises a T cell or T cell progenitor cell or NK cell;
(xi) comprises a T cell which has been modified such that its endogenous T cell receptor (TCR) is (i) not expressed, (ii) not functionally expressed, or (iii) expressed at reduced levels compared to a wild-type T cell;
(xii) is activated or stimulated to proliferate when the NKp30 variant containing agent, e.g., a CAR or bispecific engager or ADC binds to its target molecule;
(xiii) elicits cytotoxicity against cells expressing the target molecule when the NKp30 variant containing agent, optionally a CAR or bispecific engager or ADC binds to its target molecule;
(xiv) elicits cytotoxicity against cancer cells when the NKp30 variant containing agent, e.g., a CAR or bispecific engager or ADC binds to its target molecule;
(xv) it elicits increased expression of specific cytokines and/or chemokines when the NKp30 variant containing agent, e.g., a CAR or bispecific engager or ADC binds to its target molecule;
(xvi) it elicits decreased expression of specific cytokines and/or chemokines when the CAR binds to its target;
(xvii) it comprises a population of recombinant or isolated cells; or
(xviii) it comprises any combination of (i) to (xvii).

30-46. (canceled)

47. A pharmaceutical or diagnostic composition comprising:
(a) a NKp30 variant, fusion protein, conjugate, chimeric antigen receptor, binding agent drug conjugate (ADC), bispecific T cell engager, or multispecific binding polypeptide comprising said human NKp30 variant, according to claim 2,
(b) a polynucleotide or combination of polynucleotides encoding a NKp30 variant, fusion protein, conjugate, chimeric antigen receptor, binding agent drug conjugate (ADC), bispecific T cell engager, or multispecific binding polypeptide comprising said human NKp30 variant, according to claim 2; or,
(c) a cell comprising or expressing a polynucleotide or combination of polynucleotides encoding a NKp30 variant, fusion protein, conjugate, chimeric antigen receptor, binding agent drug conjugate (ADC), bispecific T cell engager, or multispecific binding polypeptide comprising said human NKp30 variant, according to claim 2;
and a pharmaceutically or diagnostically acceptable excipient or carrier.

48. A method of treatment or prophylaxis in a subject in need thereof comprising administering a pharmaceutical composition comprising:
(a) a NKp30 variant, fusion protein, conjugate, chimeric antigen receptor, binding agent drug conjugate (ADC), bispecific T cell engager, or multispecific binding polypeptide comprising said human NKp30 variant according to claim 2;
(b) a nucleic acid or vector encoding an NKp30 variant polypeptide, NKp30 variant comprising fusion protein, NKp30 variant comprising conjugate, NKp30 variant comprising bispecific T cell engager or NKp30 variant according to claim 2; or
(c) a cell, optionally an immune cell which comprises or expresses an NKp30 variant polypeptide, NKp30 variant comprising fusion protein, NKp30 variant comprising conjugate, NKp30 variant comprising bispecific T cell engager or NKp30 variant comprising CAR or ADC according to claim 2; and
a pharmaceutically acceptable carrier.

49. The method of claim 48, wherein the subject has
(i) the subject has a cancer or infectious disease condition or condition associated with cells which express or overexpress one or more NKp30 ligands, optionally B7H6 or BAT3;
(ii) the subject has a solid tumor or hematological malignancy,
(iii) the subject has one or more of ovarian, colorectal cancer, colon and rectal cancer, lung cancer, breast cancer, brain tumor, melanoma, renal cell carcinoma, bladder cancer, leukemia, lymphoma, T cell lymphoma, multiple myeloma, gastric cancer, pancreatic cancer, uterine cervical cancer, endometrial cancer, esophageal cancer, liver cancer, head and neck squamous cell carcinoma, lung cancer, kidney cancer, bladder cancer, skin cancer, urinary tract cancer, prostate cancer, choriocarcinoma, pharyngeal cancer, laryngeal cancer, tongue cancer, oral cancer, gallbladder cancer, thyroid cancer, mesothelioma, pleural tumor, arrhenoblastoma, endometrial hyperplasia, endometriosis, embryoma, fibrosarcoma, Kaposi's sarcoma, hemangioma, cavernous hemangioma, hemangioblastoma, retinoblastoma, astrocytoma, neurofibroma, oligodendroglioma, medulloblastoma, neuroblastoma, neuroglioma, rhabdomyosarcoma, glioblastoma, osteogenic sarcoma, leiomyosarcoma, thyroid sarcoma, and Wilms tumor;
(iv) the subject has one or more of head and neck cancer, brain cancer, oral cavity cancers such as Orophyarynx cancer, Nasopharynx cancer, Hypopharynx cancer, Nasal cavity cancer, paranasal sinus cancer, Larynx cancer, Lip cancer; Lung cancers such as Non-small cell carcinoma, Small cell carcinoma, Gastrointestinal Tract cancers such as Colorectal cancer, Gastric cancer, Esophageal cancer, Anal cancer, Extrahepatic Bile Duct cancer, Cancer of the Ampulla of Vater, Gastrointestinal Stromal Tumor (GIST); Liver cancers such as Liver Cell Adenoma, Hepatocellular Carcinoma; Breast cancers, Gynecologic cancers such as Cervical cancer, Ovarian cancer, Vaginal cancer, Vulvar cancer; Gestational Trophoblastic Neoplasia, Uterine cancer, Urinary Tract cancers such as Renal cancer carcinoma, Prostate cancer, Urinary Bladder cancer, Penile cancer, Urethral cancer, Urinary Bladder cancer, Neurological Tumors such as Astrocytoma and glioblastoma, Primary CNS lymphoma, Medulloblastoma, Germ Cell tumors. Retinoblastoma, Endocrine Neoplasms Thyroid cancer, Pancreatic cancers such as Islet Cell tumors, Insulinomas Glucagonomas, Pheochromocytoma, Adrenal carcinomas, Carcinoid tumors, Parathyroid carcinomas, Pineal gland neoplasms, Skin cancers such as Malignant melanoma, Squamous Cell carcinoma, Basal Cell carcinoma, Kaposi's Sarcoma, Bone cancers such as Osteoblastoma, Osteochondroma, Osteosarcoma; Connective Tissue neoplasms such as Chondroblastoma, Chondroma; Hematopoietic malignancies such as Non-Hodgkin Lymphoma B-cell lymphoma, T-cell lymphoma, Undifferentiated lymphoma; Leukemias such as Chronic Myelogenous Leukemia, Hairy Cell Leukemia, Chronic Lymphocytic Leukemia, Chronic Myelomonocytic Leukemia, Acute Myelocytic Leukemia, Acute Lymphoblastic Leukemia; Myeloproliferative Disorders such as Multiple Myeloma, Essential Thrombocythemia, Myelofibrosis with Myeloid Metaplasia, Hypereosinophilic Syndrome, Chronic Eosinophilic Leukemia, Polycythemia Vera, Hodgkin Lymphoma, Childhood Cancers such as Leukemia and Lymphomas; Brain cancers, Neuroblastoma; Wilm's Tumor (nephroblastoma), Phabdomyosarcoma; Retinoblastoma; Immunotherapeutically sensitive cancers such as melanoma, kidney cancer, leukemias, lymphomas and myelomas; breast cancer; prostate cancers; colorectal cancers; cervical cancers; ovarian cancers and lung cancers;
(v) is used for preventing and/or treating cancer, or preventing cancer reoccurrence in a subject in need thereof, optionally wherein the subject has or has had at least one cancer selected from the group consisting of suffering from one or more of ovarian, colorectal cancer, colon and rectal cancer, lung cancer, breast cancer, brain tumor, melanoma, renal cell carcinoma, bladder cancer, leukemia, lymphoma, T cell lymphoma, multiple myeloma, gastric cancer, pancreatic cancer, uterine cervical cancer, endometrial cancer, esophageal cancer, liver cancer, head and neck squamous cell carcinoma, lung cancer, kidney cancer, bladder cancer, skin cancer, urinary tract cancer, prostate cancer, choriocarcinoma, pharyngeal cancer, laryngeal cancer, tongue cancer, oral cancer, gallbladder cancer, sarcoma, leukemia, melanoma, thyroid cancer, mesothelioma, pleural tumor, arrhenoblastoma, endometrial hyperplasia, endometriosis, embryoma, fibrosarcoma, Kaposi's sarcoma, hemangioma, cavernous hemangioma, hemangioblastoma, retinoblastoma, astrocytoma, neurofibroma, oligodendroglioma, medulloblastoma, neuroblastoma, neuroglioma, rhabdomyosarcoma, glioblastoma, osteogenic sarcoma, leiomyosarcoma, thyroid sarcoma, and Wilms tumor and/or further optionally wherein the cancer has been determined to express or overexpress an NKp30 ligand, e.g., B7H6;
(vi) the method comprises administering an effective amount of a genetically modified immune cell comprising a CAR comprising an NKp30 variant according to claim 2, which is expressed on its surface; or
any combination of the foregoing.

50-54. (canceled)

55. A method of producing a variant of a receptor, optionally a variant NK receptor, further optionally an NKp30 variant that interacts with a ligand preferentially expressed or expressed at higher levels on diseased cells than normal cells, optionally cancer or virally infected cells, comprising introducing one or mutations in a receptor, optionally a NK receptor polypeptide, further optionally a NKp30 polypeptide or a nucleic acid encoding an NK receptor polypeptide, further optionally a NKp30 polypeptide, optionally by directed evolution, further optionally using yeast or phage display, and determining in one or more screens whether said variant receptor, optionally a variant NK receptor, further optionally a NKp30 variant or a CAR, bispecific T cell engager or fusion protein containing said variant receptor, variant NK receptor, or variant NKp30 polypeptide or a nucleic acid encoding any of the foregoing when expressed exhibits one or more of the following:
(i) binds to one or more of its endogenous ligands which are preferentially expressed or more highly expressed on diseased cells, e.g., cancerous or infected cells, e.g., B7H6 expressing cells, with higher affinity compared to the native receptor, e.g., NK receptor or NKp30;
(ii) retains its fast association and dissociation profile to ligand expressing cells, e.g., NK ligand expressing cells or B7H6 expressing cells, compared to the native receptor, e.g., an NK receptor polypeptide, optionally NKp30;
(iii) elicits a different cytokine profile than the native receptor, e.g., NK receptor polypeptide, optionally NKp30 or than an scFv that binds to the preferentially expressed ligand, optionally NK ligand such as B7H6 or TZ47, optionally against one or more different types of tumors which preferentially express the ligand bound by the receptor,
(iv) exhibits improved in vitro or in vivo tumor cell killing of cells which express the ligand relative to the native receptor, optionally a NK receptor, optionally against tumors or other diseased cells which express low levels of the ligand, e.g., NKp30 or compared to a scFv that binds the NK ligand, optionally TZ47 and/or
(v) binds to tumor cells or other diseased cells expressing low levels of the NK ligand, optionally B7H6 better than TZ47 and or the native receptor, optionally a NK receptor, further optionally NKp30.

56. The method of claim 55, wherein the resultant receptor variant, optionally an NK variant, further optionally NKp30 variant, is used to produce a receptor variant, optionally NK variant, further optionally NKp30 variant, comprising conjugate, bispecific T cell engager, CAR or drug conjugate or detectably labelled variant comprising the receptor variant, optionally an NK variant, further optionally NKp30 variant.

57. A method of treating a subject with an NKp30 variant, or a fusion protein, conjugate, chimeric antigen receptor, binding agent drug conjugate (ADC), bispecific T cell engager, or multispecific binding polypeptide comprising said human NKp30 variant according to claim 2 or a cell which expresses said NKp30 variant, or a fusion protein, conjugate, chimeric antigen receptor, binding agent drug conjugate (ADC), bispecific T cell engager, or multispecific binding polypeptide comprising said human NKp30 variant according to claim 2 which comprises the steps of:
(a) obtaining or having obtained a biological sample, e.g., tumor biopsy, from the subject;
(b) measuring the expression level of NKp30 ligands such as B7H6 on cells of the biological sample;
(c) determining whether the sample expresses or overexpresses NKp30 ligands; and
(d) if the subject expresses or overexpresses NKp30 ligands, administering to the subject a therapeutically or diagnostically effective amount of
(d-i) a NKp30 variant, or a fusion protein, conjugate, chimeric antigen receptor, binding agent drug conjugate (ADC), bispecific T cell engager, or multispecific binding polypeptide comprising said NKp30 variant according to claim 2
(d-ii) a polynucleotide encoding a NKp30 variant, or a fusion protein, conjugate, chimeric antigen receptor, binding agent drug conjugate (ADC), bispecific T cell engager, or multispecific binding polypeptide comprising said NKp30 variant according to claim 2, or a vector containing said polynucleotide,
(d-iii) cell which comprises or expresses a NKp30 variant, or a fusion protein, conjugate, chimeric antigen receptor, binding agent drug conjugate (ADC), bispecific T cell engager, or multispecific binding polypeptide comprising said NKp30 variant according to claim 2,
(d-iv) population of cells which comprises or expresses a NKp30 variant, or a fusion protein, conjugate, chimeric antigen receptor, binding agent drug conjugate (ADC), bispecific T cell engager, or multispecific binding polypeptide comprising said NKp30 variant according to claim 2, or
(d-v) a pharmaceutical composition comprising any of the foregoing.

58. The method of claim 57, wherein B7H6 expression is at least 1.2 times higher than B7H6 expression of cells of normal or healthy subjects, or is at least 1.5 times higher than B7H6 expression of cells of normal or healthy subjects, or is at least 1.75 times higher than the B7H6 expression of normal or healthy subjects, or is at least twice higher than the B7H6 expression of cells of normal or healthy subjects.

59. The method of claim 57, wherein the subject is suffering from cancer, optionally pancreatic cancer, testicular cancer, cervical cancer, endometrial cancer, ovarian cancer, stomach cancer, colorectal cancer, lung cancer, mesothelioma, lymphoma, tongue cancer or other cancers previously identified.

60. A treatment method according to claim 48 which further comprises administering a second agent, optionally an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an enzyme, a hormone, a toxin, a radioisotope, a compound, a small molecule, a small molecule inhibitor, a protein, a peptide, a vector, a plasmid, a viral replicon, a viral particle, a nanoparticle, a DNA molecule, an RNA molecule, an siRNA, an shRNA, a micro RNA, an oligonucleotide, or an imaging drug.
